(19) United States
(12) Patent Application Publication
(10) Pub. No.: US 2020/0105373 A1
(43) Pub. Date: Apr. 2, 2020

(54) SYSTEMS AND METHODS FOR CELLULAR ANALYSIS USING NUCLEIC ACID SEQUENCING

(71) Applicant: 10X Genomics, Inc., Pleasanton, CA (US)

(72) Inventor: Xinying Zheng, San Jose, CA (US)

(21) Appl. No.: 16/575,280

(22) Filed: Sep. 18, 2019

Related U.S. Application Data
(60) Provisional application No. 62/739,067, filed on Sep. 28, 2018.

(51) Int. Cl. G16B 30/10; C12N 15/10; C12Q 1/6874

(52) U.S. Cl. CPC G16B 30/10; G06K 19/06028; C12Q 1/6874; C12N 15/1065

(57) ABSTRACT

Template nucleic acid fragments are generated in cells or nuclei using transposase-nucleic acid complexes. Partitions are formed, each comprising a single cell or nuclei, the corresponding plurality of template nucleic acid fragments and nucleic acid barcodes comprising a corresponding common barcode sequence unique to a respective cell or nuclei. Barcoded nucleic acid fragments are generated in each partition using the barcodes and the template fragments. The barcoded fragments in each partition collectively form a pool of barcoded nucleic acid fragments. A set of alleles for each locus in a plurality of loci are identified and, for each such locus, a subset of the pool of barcoded fragments mapping to the locus are aligned to determine an allelic identity of such fragments from among the set of alleles for the locus, thereby determining a corresponding allelic distribution at each respective locus. These distributions are used to identify a structural variation.

### CROSS REFERENCE TO RELATED APPLICATION

This application claims priority to U.S. Provisional Patent Application No. 62/739,067, entitled “Methods for Cellular Analysis Using Nucleic Acid Sequencing,” filed Sep. 28, 2018, which is hereby incorporated by reference.

### BACKGROUND

A sample may be processed for various purposes, such as identification of a type of moiety within the sample. The sample may be a biological sample. Biological samples may be processed, such as for detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Biological samples in partitions may be subjected to various processes, such as chemical processes or physical processes. Samples in partitions may be subjected to heating or cooling, or chemical reactions, such as to yield species that may be qualitatively or quantitatively processed.

### SUMMARY

As part of a first procedure, there is generated, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle. A plurality of partitions is generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. A corresponding plurality of barcoded nucleic acid fragments is generated, in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. There is provided a computer system comprising at least one processor and a memory storing at least one program for execution by the at least one processor, the at least one program comprising instructions for identifying a plurality of loci, and for each respective locus in the plurality of loci, a corresponding set of alleles for the respective locus. For each respective locus in the plurality of loci, a second procedure is performed that comprises identifying a corresponding subset of the pool of barcoded nucleic acid fragments that map to the respective locus. The second procedure further uses an alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus. The second procedure further categorizes each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, where the corresponding allelic distribution includes an abundance of each allele in the corresponding set of alleles for the respective locus. The corresponding allelic distribution at each respective locus in the plurality of loci to identify a structural variation within a biological particle in the plurality of biological particles.

In some embodiments, a respective locus in the plurality of loci is biallelic and the corresponding set of alleles for the respective locus consists of a first allele and a second allele.

In some embodiments, the structural variation is a heterozygous single nucleotide polymorphism (SNP), a heterozygous single nucleotide variant (SNV), a heterozygous insert, a heterozygous deletion, or a copy number variation at a locus in the plurality of loci.

In some embodiments, the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments.

In some embodiments, the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments. In some embodiments, the plurality of loci comprises between two and 100 loci, more than 10 loci, more than 100 loci, or more than 500 loci.

In some embodiments, the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

In some embodiments, the corresponding common barcode sequence is localized to a contiguous set of oligonucleotides within the respective barcoded nucleic acid fragment. In some such embodiments, the contiguous set of oligonucleotides is an N-mer, where N is an integer selected from the set {4, . . . , 20}.

In some embodiments, identifying the plurality of loci comprises retrieving the plurality of loci and each corresponding set of alleles from a lookup table, file or data structure.

In some embodiments, the pool of barcoded nucleic acid fragments is used to identify the plurality of loci, and for each respective locus in the plurality of loci, the corresponding set of alleles for the respective locus.

In some embodiments, the alignment is a local alignment (e.g., a Smith-Waterman alignment) that aligns the respective barcoded nucleic acid fragment to a reference sequence (e.g., all or portion of a reference genome) using a scoring system that (i) penalizes a mismatch between a nucleotide in the respective barcoded nucleic acid fragment and a corresponding nucleotide in the reference sequence in accordance with a substitution matrix and (ii) penalizes a gap introduced into an alignment of the respective barcoded nucleic acid fragment and the reference sequence.

In some embodiments, the method further comprises removing from the pool of barcoded nucleic acid fragments one or more barcoded nucleic acid fragments that do not overlay any loci in the plurality of loci.

In some embodiments, the plurality of loci include one or more loci on a first chromosome and one or more loci on a second chromosome other than the first chromosome.

In some embodiments, each partition in the plurality of partitions is a droplet or a well. In some embodiments, each biological particle in the plurality of biological particles is a single cell nuclei harvested from its cell. In some embodiments, biological particle in the plurality of biological particles is a single cell.

In some embodiments, the transposase molecule is a native Tn5 transposase, a mutated hyperactive Tn5 transposase, or a Mu transposase. In some embodiments, the transposon end nucleic acid molecule is a Tn5 or modified Tn5 transposon end sequence.

In some embodiments, the corresponding plurality of nucleic acid barcode molecules are attached to a solid or semi-solid particle (e.g., a gel bead).

In some embodiments, the biological sample is from a single subject. In some embodiments, the biological sample is from a plurality of subjects.

In some embodiments, a corresponding genotypic data structure is determined for each biological particle in the plurality of biological particles using the disclosed systems and methods, thereby constructing a plurality of genotypic data structures and, further, the corresponding genotypic data structure for each biological particle in the plurality of particles is used to segregate the plurality of biological particles to determine a property (e.g., absence or presence of a disease, a stage of a disease, a cell type, an identification of a species) of each biological particle in the plurality of biological particles.

In some embodiments, the plurality of loci are in a reference genome (e.g., a human reference genome, a mitochondrial genome).

Another aspect of the present disclosure provides an electronic device, comprising one or more processors, memory, and one or more programs. The one or more programs are stored in the memory and are configured to be executed by the one or more processors. The one or more programs are for identifying a structural variation. The one or more programs include instructions for obtaining, in electronic form, a pool of barcoded nucleic acid fragments by a first procedure that comprises (i) generating, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle. Further, a plurality of partitions is generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. A corresponding plurality of barcoded nucleic acid fragments is generated in each respective partition in the plurality of partitions using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. A plurality of loci is identified, and for each respective locus in the plurality of loci, a corresponding set of alleles for the respective locus is identified.

For each respective locus in the plurality of loci, a second procedure is performed in which a corresponding subset of the pool of barcoded nucleic acid fragments that map to the respective locus is identified. An alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus. Each respective barcoded nucleic acid fragment in the corresponding subset of the pool of barcoded nucleic acid fragments is categorized by the allelic identity and barcode identity of the respective barcoded nucleic acid fragment, thereby determining a corresponding allelic distribution at each respective locus in the plurality of loci, for each biological particle in the plurality of biological particles. The corresponding allelic distribution includes an abundance of each allele in the corresponding set of alleles for the respective locus. The corresponding allelic distribution at each respective locus in the plurality of loci is used to identify a structural variation within a biological particle in the plurality of biological particles.

Another aspect of the present disclosure provides a computer readable storage medium storing one or more programs. The one or more programs comprise instructions, that when executed by an electronic device with one or more processors and a memory, cause the electronic device to identify a structural variation by a method comprising obtaining, in electronic form, a pool of barcoded nucleic acid fragments by a first procedure that comprises generating, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle. A plurality of partitions is generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. A corresponding plurality of barcoded nucleic acid fragments is generated in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. A plurality of loci, and for each respective locus in the plurality of loci, a corresponding set of alleles for the respective locus is identified.

For each respective locus in the plurality of loci, a second procedure is performed that comprises identifying a corresponding subset of the pool of barcoded nucleic acid fragments that map to the respective locus and using an alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus. Each respective barcoded nucleic acid fragment in the corresponding subset of the pool of barcoded nucleic acid fragments is categorized by the allelic identity and barcode identity of the respective barcoded nucleic acid fragment, thereby determining a corresponding allelic distribution at each respective locus in the plurality of loci, for each biological particle in the plurality of biological particles, where the corresponding allelic distribution includes an abundance of each allele in the corresponding set of alleles for the respective locus. The corresponding allelic distribution at each respective locus in the plurality of loci to is used to identify a structural variation within a biological particle in the plurality of biological particles.

Another aspect of the present disclosure is a physiological state determination method in which a pool of barcoded nucleic acid fragments is generated by a first procedure that comprises generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle. Further, a plurality of partitions is generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. A corresponding plurality of barcoded nucleic acid fragments, in each respective partition in the plurality of partitions is generated, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. A computer system comprising at least one processor and a memory storing at least one program for execution by the at least one processor, the at least one program comprising instructions for performing a second procedure is provided. In the second procedure, for each respective locus in a plurality of loci, a corresponding subset of the pool of barcoded nucleic acid fragments that map to the respective locus is identified. Further, an alignment of each respective barcoded nucleic acid fragment in the corresponding subset of the pool of barcoded nucleic acid fragments is done to determine an allelic identity of each respective barcoded nucleic acid fragment from among a corresponding set of alleles for the respective locus. Further still, each respective barcoded nucleic acid fragment in the corresponding subset of the pool of barcoded nucleic acid fragments is categorized by the allelic identity and barcode identity of the respective barcoded nucleic acid fragment, thereby determining a corresponding allelic distribution at each respective locus in the plurality of loci, for each biological particle in the plurality of biological particles. The corresponding allelic distribution includes an abundance of each allele in the corresponding set of alleles for the respective locus. The corresponding allelic distribution at each respective locus in the plurality of loci is used to determine the physiological state of the single test subject.

In some embodiments, a respective locus in the plurality of loci is biallelic and the corresponding set of alleles for the respective locus consists of a first allele and a second allele.

In some embodiments, the respective locus includes a heterozygous single nucleotide polymorphism (SNP), a heterozygous single nucleotide variant (SNV), a heterozygous insert, a heterozygous deletion, or a copy number variation.

In some embodiments, the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments.

In some embodiments, the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments.

In some embodiments, the plurality of loci comprises between two and 100 loci, more than 10 loci, more than 100 loci, or more than 500 loci.

In some embodiments, the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

In some embodiments, the corresponding common barcode sequence is localized to a contiguous set of oligonucleotides within the respective barcoded nucleic acid fragment.

In some embodiments, plurality of loci are identified by retrieving the plurality of loci and each corresponding set of alleles from a lookup table, file or data structure. In some alternative embodiments, the pool of barcoded nucleic acid fragments is used to identify the plurality of loci, and for each respective locus in the plurality of loci, the corresponding set of alleles for the respective locus.

In some embodiments, the alignment is a local alignment (e.g., a Smith-Waterman alignment) that aligns the respective barcoded nucleic acid fragment to a reference sequence using a scoring system that (i) penalizes a mismatch between a nucleotide in the respective barcoded nucleic acid fragment and a corresponding nucleotide in the reference sequence (e.g., all or portion of a reference genome) in accordance with a substitution matrix and (ii) penalizes a gap introduced into an alignment of the respective barcoded nucleic acid fragment and the reference sequence.

In some embodiments, the plurality of loci include one or more loci on a first chromosome and one or more loci on a second chromosome other than the first chromosome.

In some embodiments, each partition in the plurality of partitions is a droplet or a well.

In some embodiments, each biological particle in the plurality of biological particles is a single cell nuclei harvested from its cell. In some embodiments, each biological particle in the plurality of biological particles is a single cell.

In some embodiments, the transposase molecule is a native Tn5 transposase, a mutated hyperactive Tn5 transposase, or a Mu transposase. In some embodiments, the transposon end nucleic acid molecule is a Tn5 or modified Tn5 transposon end sequence.

In some embodiments the corresponding plurality of nucleic acid barcode molecules are attached to a solid or semi-solid particle (e.g., a gel bead). In some embodiments, the physiological state is absence or presence of a disease. In some embodiments, the physiological state is a stage of a disease. In some embodiments, the plurality of loci are in a reference genome (e.g., a human reference genome). In some embodiments, the reference genome is a mitochondrial genome.

In some embodiments, the corresponding allelic distribution at each respective locus in the plurality of loci is inputted into a classifier and the classifier, responsive to this inputting, provides the physiological state of the single test subject. In some embodiments, the classifier is a multinomial classifier that provides a plurality of likelihoods, where each respective likelihood in the plurality of likelihoods is a likelihood that the single test subject has a corresponding physiological state in a plurality of physiological states. In some embodiments, each physiological state in the plurality of physiological states is a cancer class in a plurality of cancer classes. In some embodiments, the classifier is a multivariate logistic regression algorithm, a neural network algorithm, or a convolutional neural network algorithm. In some embodiments, the classifier is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

Another aspect of the present disclosure is an electronic device comprising one or more processors, memory, and one or more programs. The one or more programs are stored in the memory and are configured to be executed by the one or more processors. The one or more programs are for determining a physiological state. The one or more programs include instructions for obtaining, in electronic form, a pool of barcoded nucleic acid fragments generated by a first procedure that comprises generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle. Further, a plurality of partitions are generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. Further still, a corresponding plurality of barcoded nucleic acid fragments is generated in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. Further still, a second procedure is performed for each respective locus in a plurality of loci that comprises identifying a corresponding subset of the pool of barcoded nucleic acid fragments that map to the respective locus, using an alignment of each respective barcoded nucleic acid fragment in the corresponding subset of the pool of barcoded nucleic acid fragments to determine an allelic identity of each respective barcoded nucleic acid fragment from among a corresponding set of alleles for the respective locus, and categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, where the corresponding allelic distribution includes an abundance of each allele in the corresponding set of alleles for the respective locus. The corresponding allelic distribution at each respective locus in the plurality of loci is used to determine the physiological state of the single test subject.

Another aspect of the present disclosure provides a computer readable storage medium storing one or more programs. The one or more programs comprise instructions, which when executed by an electronic device with one or more processors and a memory, cause the electronic device to determine a physiological by a method comprising obtaining, in electronic form, a pool of barcoded nucleic acid fragments generated by a first procedure. The first procedure comprises (i) generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle, (ii) generating a plurality of partitions, where each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. The first procedure further comprises (iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. A second procedure, for each respective locus in a plurality of loci, that comprises i) identifying a corresponding subset of the pool of barcoded nucleic acid fragments that map to the respective locus, ii) using an alignment of each respective barcoded nucleic acid fragment in the corresponding subset of the pool of barcoded nucleic acid fragments to determine an allelic identity of each respective barcoded nucleic acid fragment from among a corresponding set of alleles for the respective locus, and iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles. The corresponding allelic distribution includes an abundance of each allele in the corresponding set of alleles for the respective locus. Further still, the corresponding allelic distribution at each respective locus in the plurality of loci is used to determine the physiological state of the single test subject.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIGS. 7A and 7B. FIG. 7A shows a cross-section view of another example of a microfluidic channel structure with a geometric feature for controlled partitioning. FIG. 7B shows a perspective view of the channel structure of FIG. 7A.

FIG. 8 illustrates an example of a barcode carrying bead.

FIG. 9 shows the results from a single cell sequencing experiment, in which multiple single nucleotide polymorphisms (SNPs) are detected in the ACTB gene from human GM12878 cells.

FIG. 10 shows the results from a single cell sequencing experiment, where single nucleotide variants (SNV) are used to distinguish human cells from mouse cells.

FIG. 11 shows the accuracy of variant detection in GM12878 cells using the disclosed single cell analysis methods.

FIGS. 12A and 12B show the results from a single cell sequencing experiment, where sequences from two subjects (Donor 1 and Donor 2) were mixed and analyzed for SNVs, such that SNVs specific to each individual were identified. FIG. 12A grey scale codes for a particular allelic position (Chr1.564995) that contains a SNV specific to Donor 2, and therefore shows how the cells from Donor 2 cluster together in the bottom right portion of the graph. FIG. 12B grey scale codes for a particular allelic position (Chr1.624866) that contains a SNV specific to Donor 1, and therefore shows how the cells from Donor 1 cluster together in the upper left hand portion of the graph.

FIG. 13 shows an example of detecting a SNP in a mitochondria region from single cell sequencing data obtained from human cells.

FIGS. 14A and 14B show analysis of single cell sequencing data from human and mouse cells having about 40% measured barcoded nucleic acid fragments of mitochondrial origin. FIG. 14A shows analysis of all measured barcoded nucleic acid fragments. FIG. 14B shows analysis of measured barcoded nucleic acid fragments having mitochondrial origin only.

FIGS. 15A and 15B show analysis of single cell sequencing data from human and mouse cells having about 10% measured barcoded nucleic acid fragments of mitochondrial origin. FIG. 15A shows analysis of all measured barcoded nucleic acid fragments. FIG. 15B shows analysis of measured barcoded nucleic acid fragments having mitochondrial origin only.

FIG. 16 shows a computer system that is programmed or otherwise configured to implement methods provided herein.

FIGS. 17A, 17B and 17C illustrate exemplary compositions for use in the transposition and barcoding of nucleic acid molecules. FIG. 17A illustrates an exemplary transposase-nucleic acid complex showing a transposase, a first partially double-stranded oligonucleotide comprising a first adapter sequence, and a second partially double-stranded oligonucleotide comprising a second adapter sequence. FIGS. 17B-17C illustrate exemplary barcode molecules.

FIG. 18 illustrates an exemplary method for generating barcoded, adapter-flanked nucleic acid fragments using bulk tagmentation and barcoding via ligation in partitions.

FIG. 19 illustrates an exemplary method for generating barcoded, adapter-flanked nucleic acid fragments using in-partition tagmentation and barcoding via ligation.

FIG. 20 illustrates an exemplary method for generating barcoded, adapter-flanked nucleic acid fragments using bulk tagmentation and barcoding via linear amplification in partitions.

FIG. 21 illustrates an exemplary method for generating barcoded, adapter-flanked nucleic acid fragments using in-partition tagmentation and barcoding via linear amplification.

FIG. 22 illustrates an exemplary bulk processing scheme to generate barcoded fragments suitable for next generation sequencing analysis.

FIG. 23 illustrates a workflow schematic detailing the process flow and input data structures for variant identification in accordance with some embodiments of the present disclosure.

FIGS. 24A, 24B, 24C, 24D, and 24E illustrate performing structural variation identification in accordance with some embodiments of the present disclosure, in which optional steps are illustrated by dashed line boxes.

FIG. 25 shows the results from a single cell sequencing experiment, where sequences from the peripheral blood mononuclear cells (PBMCs) cells of two subjects (Donor 1 and Donor 2) were mixed and analyzed for SNVs, such that SNVs specific to each individual were identified in accordance with the disclosed methods.

FIGS. 26A and 26B respectively provide the allelic distribution for the locus of the LMB2 gene across the cells from Donor 2 (reference allele, FIG. 26A) and Donor 2 (alternative allele, FIG. 26B) that clustered into t-SNE cluster 3702 of FIG. 25 in accordance with the disclosed methods.

### DETAILED DESCRIPTION

In eukaryotic genomes, chromosomal DNA winds itself around histone proteins (i.e., “nucleosomes”), thereby forming a complex known as chromatin. The tight or loose packaging of chromatin contributes to the control of gene expression. Tightly packed chromatin (“closed chromatin”) is usually not permissive for gene expression while more loosely packaged, accessible regions of chromatin (“open chromatin”) is associated with the active transcription of gene products. Methods for probing genome-wide DNA accessibility have proven extremely effective in identifying regulatory elements across a variety of cell types and quantifying changes that lead to both activation or repression of gene expression.

One such method is the Assay for Transposase Accessible Chromatin with high-throughput sequencing (ATAC-seq). The ATAC-seq method probes DNA accessibility with an artificial transposon, which inserts specific sequences into accessible regions of chromatin. Because the transposase preferentially inserts sequences into accessible regions of chromatin not bound by transcription factors and/or nucleosomes, sequencing reads can be used to infer regions of increased chromatin accessibility. For a description of exemplary ATAC-seq methodologies, compositions, and systems, including single cell analyses, see, e.g., U.S. Pat. No. 10,059,989 and U.S. Pat. Pub. 20180340171, which are both hereby incorporated by reference in their entireties.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about a nucleic acid fragment. A barcode can be part of a nucleic acid fragment. A barcode can be independent of a nucleic acid fragment. A barcode can be a tag attached to a nucleic acid fragment (e.g., nucleic acid molecule). A barcode may be unique. Barcodes can have a variety of different formats. For example, barcodes can include: polynucleotide barcodes; random nucleic acid sequences; and synthetic nucleic acid sequences. A barcode can be attached to a nucleic acid fragment in a reversible or irreversible manner. A barcode can be added to, for example, a fragment of a deoxyribonucleic acid (DNA) before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The terms “sample” and “biological sample” are interchangeably used herein. The sample may be a cell sample. The sample may be a cell line or cell culture sample. The sample can include one or more cells. The sample can include one or more microbes. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab.

The term “biological particle,” as used herein, generally refers to a discrete biological system from a biological sample. The biological particle may be a cell or derivative of a cell. The biological particle may be a cell nucleus that has been removed from its cell. Such a cell nucleic may be associated with one or more biological components such as mitochrondial nucleic acids from the corresponding cell. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be obtained from a tissue or liquid biological sample (e.g., blood) of a subject. The biological particle may be a hardened cell. Such hardened cell may or may not include a cell wall or cell membrane. The biological particle may include one or more constituents of a cell, but may not include other constituents of the cell. An example of such constituents is a nucleus. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

Systems and Methods for Sample Compartmentalization.

In an aspect, the systems and methods described herein provide for the compartmentalization, depositing, or partitioning of one or more particles (e.g., biological particles, macromolecular constituents of biological particles, beads, reagents, etc.) into discrete compartments or partitions (referred to interchangeably herein as partitions), where each partition maintains separation of its own contents from the contents of other partitions. The partition can be a droplet in an emulsion. A partition may comprise one or more other partitions.

A partition may include one or more particles. A partition may include one or more types of particles. For example, a partition of the present disclosure may comprise one or more biological particles and/or macromolecular constituents thereof. A partition may comprise one or more gel beads. A partition may comprise one or more cell beads. A partition may include a single gel bead, a single cell bead, or both a single cell bead and single gel bead. A partition may include one or more reagents. Alternatively, a partition may be unoccupied. For example, a partition may not comprise a bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described elsewhere herein. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can be droplets of a first phase within a second phase, where the first and second phases are immiscible. For example, the partitions can be droplets of aqueous fluid within a non-aqueous continuous phase (e.g., oil phase). In another example, the partitions can be droplets of a non-aqueous fluid within an aqueous phase. In some examples, the partitions may be provided in a water-in-oil emulsion or oil-in-water emulsion. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual particles to discrete partitions may in one non-limiting example be accomplished by introducing a flowing stream of particles in an aqueous fluid into a flowing stream of a non-aqueous fluid, such that droplets are generated at the junction of the two streams. Fluid properties (e.g., fluid flow rates, fluid viscosities, etc.), particle properties (e.g., volume fraction, particle size, particle concentration, etc.), microfluidic architectures (e.g., channel geometry, etc.), and other parameters may be adjusted to control the occupancy of the resulting partitions (e.g., number of biological particles per partition, number of beads per partition, etc.). For example, partition occupancy can be controlled by providing the aqueous stream at a certain concentration and/or flow rate of particles. To generate single biological particle partitions, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, DNA, cell or cellular material). In some embodiments, the various parameters (e.g., fluid properties, particle properties, microfluidic architectures, etc.) may be selected or adjusted such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

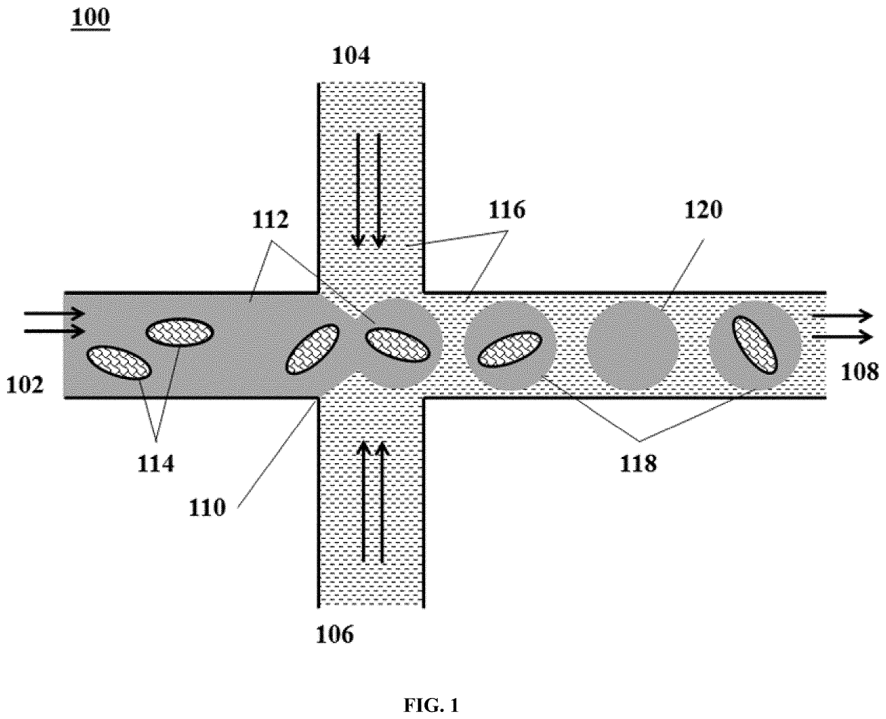

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying particles (e.g., biological particles, cell beads, and/or gel beads) that meet at a channel junction. Fluid may be directed to flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

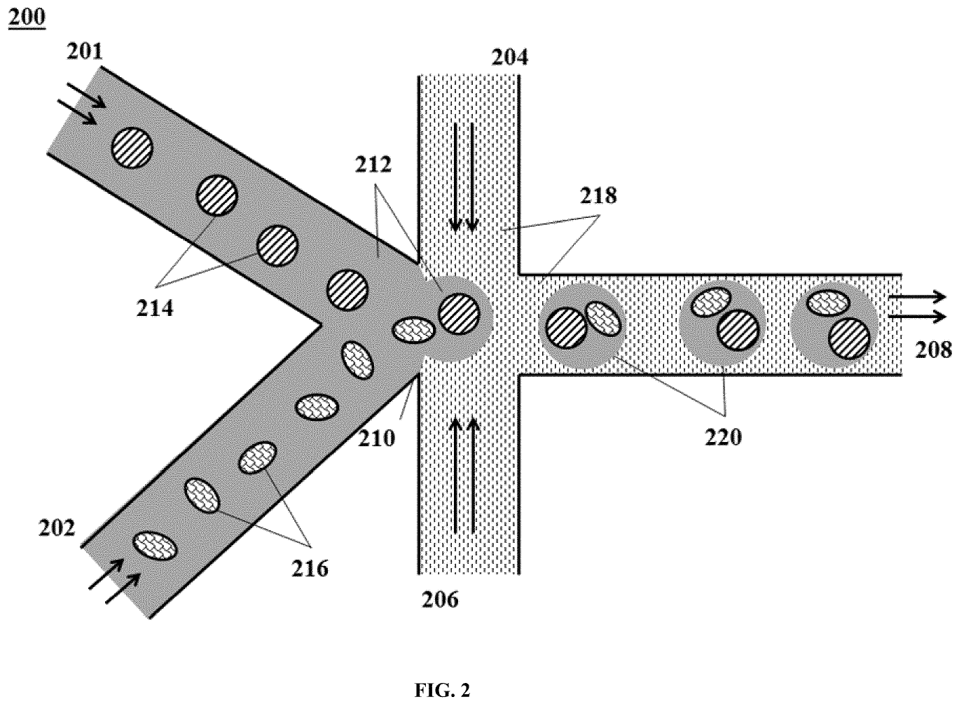

As will be appreciated, the above-described occupancy rates are also applicable to partitions that include both biological particles and additional reagents, including, but not limited to, microcapsules or beads (e.g., gel beads) carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), mechanical stimuli, or a combination thereof.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca2+ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, hyaluronic acid, collagen, fibrin, gelatin, or elastin. The polymer or gel may comprise any other polymer or gel.

The polymer or gel may be functionalized to bind to targeted analytes, such as nucleic acids, proteins, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

Beads.

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 10 nanometers (nm), 100 nm, 500 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 10 nm, 100 nm, 500 nm, lμm, 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, linear polymers), nucleic acid molecules (e.g., oligonucleotides), primers, and other entities. In some cases, the covalent bonds can be carbon-carbon bonds, thioether bonds, or carbon-heteroatom bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

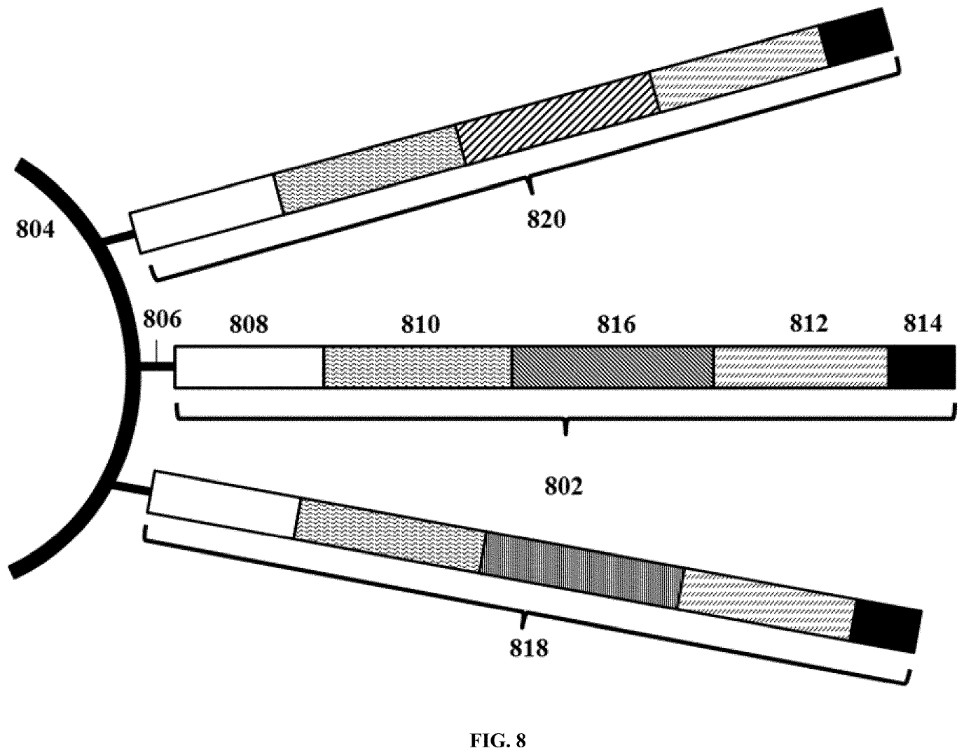

FIG. 8 illustrates an example of a barcode carrying bead. A nucleic acid molecule 802, such as an oligonucleotide, can be coupled to a bead 804 by a releasable linkage 806, such as, for example, a disulfide linker. The same bead 804 may be coupled (e.g., via releasable linkage) to one or more other nucleic acid molecules 818, 820. The nucleic acid molecule 802 may be or comprise a barcode. As noted elsewhere herein, the structure of the barcode may comprise a number of sequence elements. The nucleic acid molecule 802 may comprise a functional sequence 808 that may be used in subsequent processing. For example, the functional sequence 808 may include one or more of a sequencer specific flow cell attachment sequence (e.g., a P5 sequence for Illumina® sequencing systems) and a sequencing primer sequence (e.g., a R1 primer for Illumina® sequencing systems). The nucleic acid molecule 802 may comprise a barcode sequence 810 for use in barcoding the sample (e.g., DNA, RNA, protein, etc.). In some cases, the barcode sequence 810 can be bead-specific such that the barcode sequence 810 is common to all nucleic acid molecules (e.g., including nucleic acid molecule 802) coupled to the same bead 804. Alternatively or in addition, the barcode sequence 810 can be partition-specific such that the barcode sequence 810 is common to all nucleic acid molecules coupled to one or more beads that are partitioned into the same partition. The nucleic acid molecule 802 may comprise a specific priming sequence 812, such as an mRNA specific priming sequence (e.g., poly-T sequence), a targeted priming sequence, and/or a random priming sequence. The nucleic acid molecule 802 may comprise an anchoring sequence 814 to ensure that the specific priming sequence 812 hybridizes at the sequence end (e.g., of the mRNA). For example, the anchoring sequence 814 can include a random short sequence of nucleotides, such as a 1-mer, 2-mer, 3-mer or longer sequence, which can ensure that a poly-T segment is more likely to hybridize at the sequence end of the poly-A tail of the mRNA.

The nucleic acid molecule 802 may comprise a unique molecular identifying sequence 816 (e.g., unique molecular identifier (UMI)). In some cases, the unique molecular identifying sequence 816 may comprise from about 5 to about 8 nucleotides. Alternatively, the unique molecular identifying sequence 816 may compress less than about 5 or more than about 8 nucleotides. The unique molecular identifying sequence 816 may be a unique sequence that varies across individual nucleic acid molecules (e.g., 802, 818, 820, etc.) coupled to a single bead (e.g., bead 804). In some cases, the unique molecular identifying sequence 816 may be a random sequence (e.g., such as a random N-mer sequence). For example, the UMI may provide a unique identifier of the starting mRNA molecule that was captured, in order to allow quantitation of the number of original expressed RNA. As will be appreciated, although FIG. 8 shows three nucleic acid molecules 802, 818, 820 coupled to the surface of the bead 804, an individual bead may be coupled to any number of individual nucleic acid molecules, for example, from one to tens to hundreds of thousands or even millions of individual nucleic acid molecules. The respective barcodes for the individual nucleic acid molecules can comprise both common sequence segments or relatively common sequence segments (e.g., 808, 810, 812, etc.) and variable or unique sequence segments (e.g., 816) between different individual nucleic acid molecules coupled to the same bead.

In operation, a biological particle (e.g., cell, DNA, RNA, etc.) can be co-partitioned along with a barcode bearing bead 804. The barcoded nucleic acid molecules 802, 818, 820 can be released from the bead 804 in the partition. By way of example, in the context of analyzing sample RNA, the poly-T segment (e.g., 812) of one of the released nucleic acid molecules (e.g., 802) can hybridize to the poly-A tail of an mRNA molecule. Reverse transcription may result in a cDNA transcript of the mRNA, but which transcript includes each of the sequence segments 808, 810, 816 of the nucleic acid molecule 802. Because the nucleic acid molecule 802 comprises an anchoring sequence 814, it will more likely hybridize to and prime reverse transcription at the sequence end of the poly-A tail of the mRNA. Within any given partition, all of the cDNA transcripts of the individual mRNA molecules may include a common barcode sequence segment 810. However, the transcripts made from the different mRNA molecules within a given partition may vary at the unique molecular identifying sequence 812 segment (e.g., UMI segment). Beneficially, even following any subsequent amplification of the contents of a given partition, the number of different UMIs can be indicative of the quantity of mRNA originating from a given partition, and thus from the biological particle (e.g., cell). As noted above, the transcripts can be amplified, cleaned up and sequenced to identify the sequence of the cDNA transcript of the mRNA, as well as to sequence the barcode segment and the UMI segment. While a poly-T primer sequence is described, other targeted or random priming sequences may also be used in priming the reverse transcription reaction. Likewise, although described as releasing the barcoded oligonucleotides into the partition, in some cases, the nucleic acid molecules bound to the bead (e.g., gel bead) may be used to hybridize and capture the mRNA on the solid phase of the bead, for example, in order to facilitate the separation of the RNA from other cell contents.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead. In some cases, any combination of stimuli may trigger degradation of a bead. For example, a change in pH may enable a chemical agent (e.g., DTT) to become an effective reducing agent.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, where DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

Reagents. In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

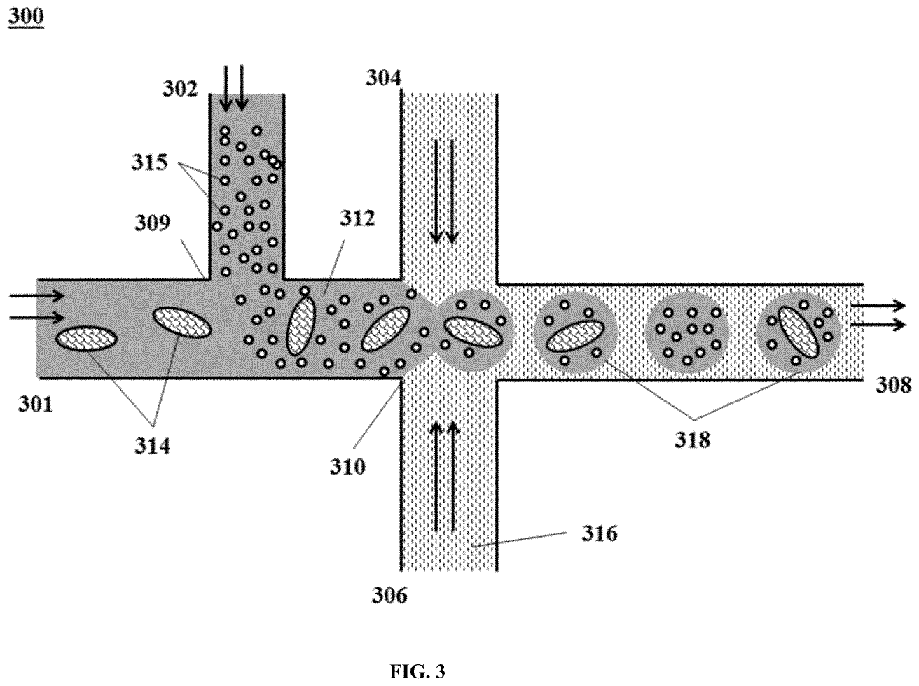

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particles' contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

Alternatively or in addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particles, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). The nucleic acid barcode sequences can include from about 6 to about 20, 30, 40, 50, 60, 70, 80, 90, 100 or more nucleotides. In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

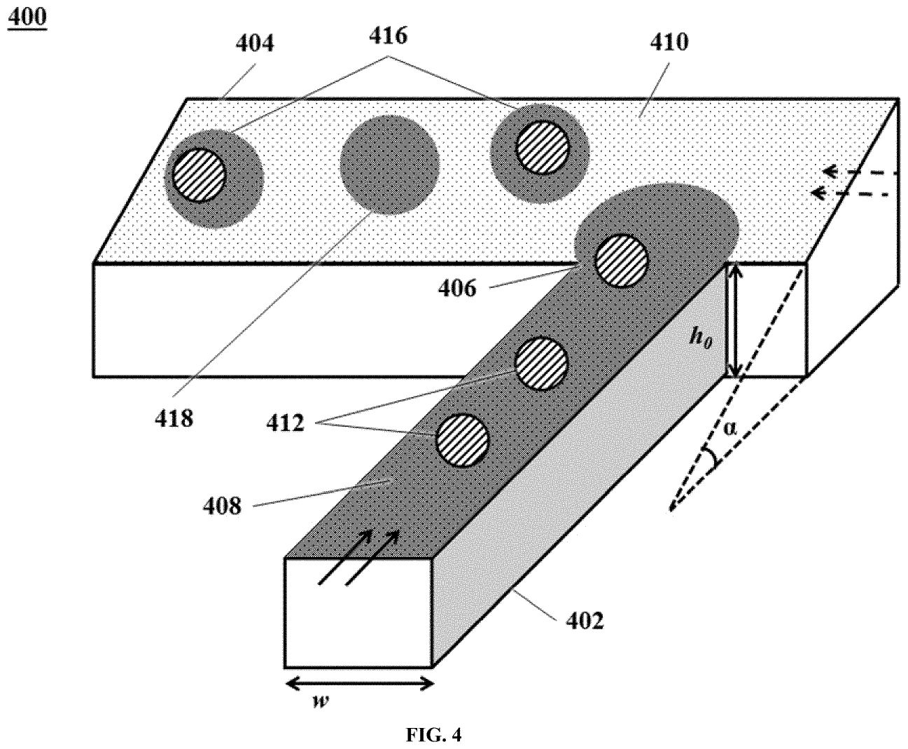

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the junction 406 where the aqueous fluid 408 and the second fluid 410 meet, droplets can form based on factors such as the hydrodynamic forces at the junction 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the junction 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the junction 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the junction 406 may have certain geometric features that at least partly determine the sizes of the droplets formed by the channel structure 400. The channel segment 402 can have a height, h₀ and width, w, at or near the junction 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the junction 406 can be inclined at an expansion angle, α. The expansion angle, α, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, Rd, may be predicted by the following equation for the aforementioned geometric parameters of h₀, w and α:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 μm, h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μall) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

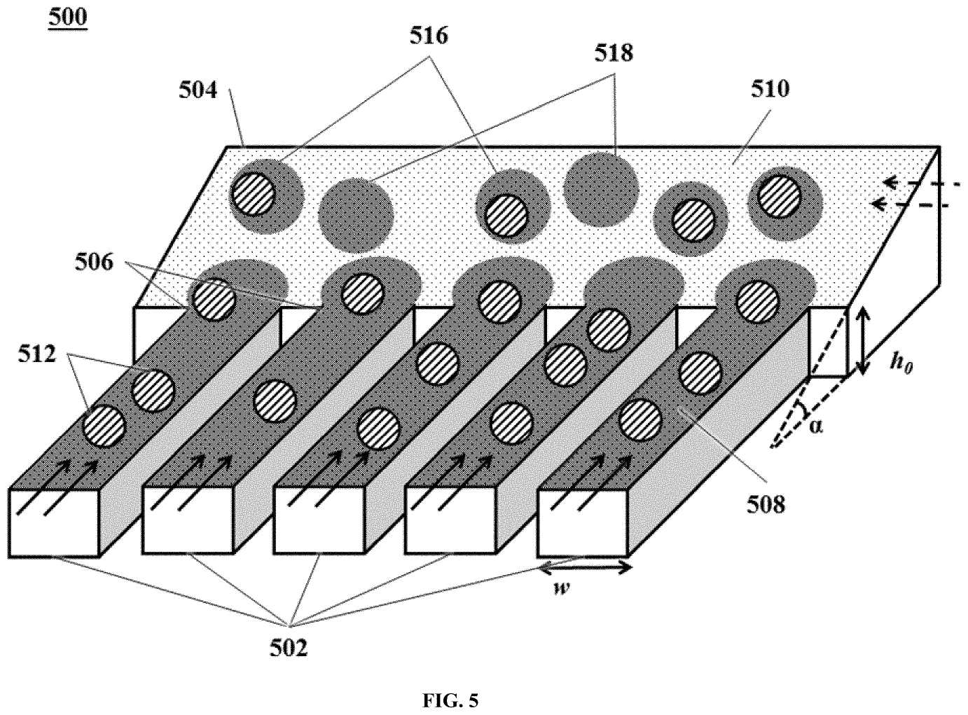

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctions. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the junction where the aqueous fluid 508 and the second fluid 510 meet, droplets can form based on factors such as the hydrodynamic forces at the junction, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctions 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and α, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

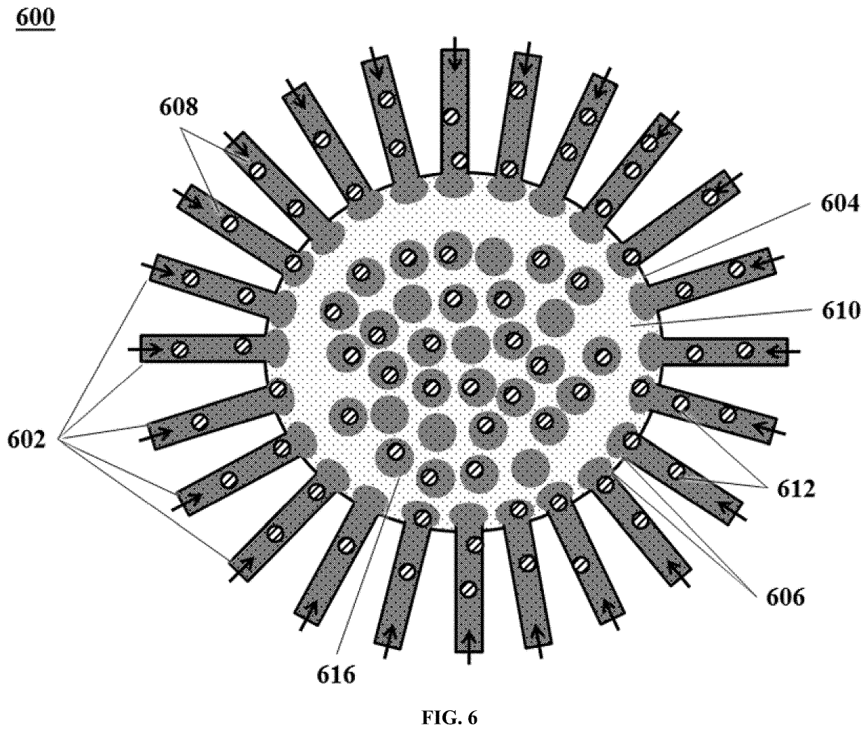

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 4 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctions. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the junction where the aqueous fluid 608 and the second fluid 610 meet, droplets can form based on factors such as the hydrodynamic forces at the junction, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctions 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, a (not shown in FIG. 6) at or near each channel junction. Each channel segment of the plurality of channel segments 602 may have a width, w, and a height, h₀, at or near the channel junction. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

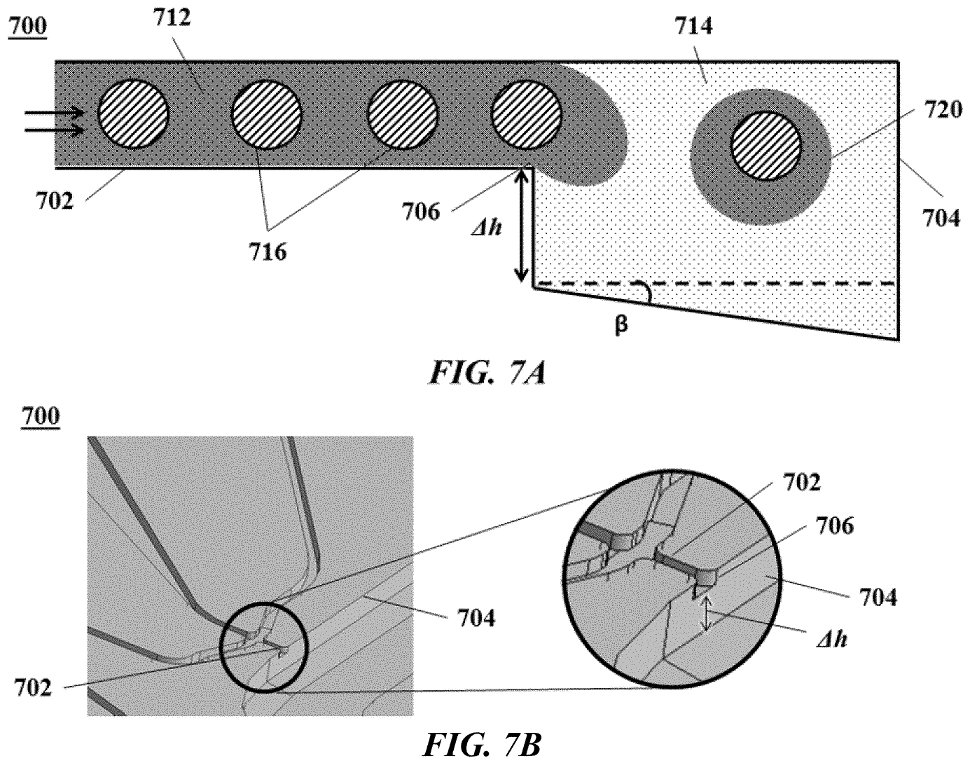

FIG. 7A shows a cross-section view of another example of a microfluidic channel structure with a geometric feature for controlled partitioning. A channel structure 700 can include a channel segment 702 communicating at a channel junction 706 (or intersection) with a reservoir 704. In some instances, the channel structure 700 and one or more of its components can correspond to the channel structure 100 and one or more of its components. FIG. 7B shows a perspective view of the channel structure 700 of FIG. 7A.

An aqueous fluid 712 comprising a plurality of particles 716 may be transported along the channel segment 702 into the junction 706 to meet a second fluid 714 (e.g., oil, etc.) that is immiscible with the aqueous fluid 712 in the reservoir 704 to create droplets 720 of the aqueous fluid 712 flowing into the reservoir 704. At the junction 706 where the aqueous fluid 712 and the second fluid 714 meet, droplets can form based on factors such as the hydrodynamic forces at the junction 706, relative flow rates of the two fluids 712, 714, fluid properties, and certain geometric parameters (e.g., Δh, etc.) of the channel structure 700. A plurality of droplets can be collected in the reservoir 704 by continuously injecting the aqueous fluid 712 from the channel segment 702 at the junction 706.

A discrete droplet generated may comprise one or more particles of the plurality of particles 716. As described elsewhere herein, a particle may be any particle, such as a bead, cell bead, gel bead, biological particle, macromolecular constituents of biological particle, or other particles. Alternatively, a discrete droplet generated may not include any particles.

In some instances, the aqueous fluid 712 can have a substantially uniform concentration or frequency of particles 716. As described elsewhere herein (e.g., with reference to FIG. 4), the particles 716 (e.g., beads) can be introduced into the channel segment 702 from a separate channel (not shown in FIG. 7). The frequency of particles 716 in the channel segment 702 may be controlled by controlling the frequency in which the particles 716 are introduced into the channel segment 702 and/or the relative flow rates of the fluids in the channel segment 702 and the separate channel. In some instances, the particles 716 can be introduced into the channel segment 702 from a plurality of different channels, and the frequency controlled accordingly. In some instances, different particles may be introduced via separate channels. For example, a first separate channel can introduce beads and a second separate channel can introduce biological particles into the channel segment 702. The first separate channel introducing the beads may be upstream or downstream of the second separate channel introducing the biological particles.

In some instances, the second fluid 714 may not be subjected to and/or directed to any flow in or out of the reservoir 704. For example, the second fluid 714 may be substantially stationary in the reservoir 704. In some instances, the second fluid 714 may be subjected to flow within the reservoir 704, but not in or out of the reservoir 704, such as via application of pressure to the reservoir 704 and/or as affected by the incoming flow of the aqueous fluid 712 at the junction 706. Alternatively, the second fluid 714 may be subjected and/or directed to flow in or out of the reservoir 704. For example, the reservoir 704 can be a channel directing the second fluid 714 from upstream to downstream, transporting the generated droplets.

The channel structure 700 at or near the junction 706 may have certain geometric features that at least partly determine the sizes and/or shapes of the droplets formed by the channel structure 700. The channel segment 702 can have a first cross-section height, h1, and the reservoir 704 can have a second cross-section height, h2. The first cross-section height, h1, and the second cross-section height, h2, may be different, such that at the junction 706, there is a height difference of Δh. The second cross-section height, h2, may be greater than the first cross-section height, h1. In some instances, the reservoir may thereafter gradually increase in cross-section height, for example, the more distant it is from the junction 706. In some instances, the cross-section height of the reservoir may increase in accordance with expansion angle, β, at or near the junction 706. The height difference, Δh, and/or expansion angle, β, can allow the tongue (portion of the aqueous fluid 712 leaving channel segment 702 at junction 706 and entering the reservoir 704 before droplet formation) to increase in depth and facilitate decrease in curvature of the intermediately formed droplet. For example, droplet size may decrease with increasing height difference and/or increasing expansion angle.

The height difference, Δh, can be at least about 1 μm. Alternatively, the height difference can be at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500 μm or more. Alternatively, the height difference can be at most about 500, 400, 300, 200, 100, 90, 80, 70, 60, 50, 45, 40, 35, 30, 25, 20, 19, 18, 17, 16, 15, 14, 13, 12, 11, 10, 9, 8, 7, 6, 5, 4, 3, 2, 1 μm or less. In some instances, the expansion angle, β, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less.

In some instances, the flow rate of the aqueous fluid 712 entering the junction 706 can be between about 0.04 microliters (μL)/minute (min) and about 40 μL/min. In some instances, the flow rate of the aqueous fluid 712 entering the junction 706 can be between about 0.01 microliters (μL)/minute (min) and about 100 μL/min. Alternatively, the flow rate of the aqueous fluid 712 entering the junction 706 can be less than about 0.01 μL/min. Alternatively, the flow rate of the aqueous fluid 712 entering the junction 706 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 712 entering the junction 706. The second fluid 714 may be stationary, or substantially stationary, in the reservoir 704. Alternatively, the second fluid 714 may be flowing, such as at the above flow rates described for the aqueous fluid 712.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

While FIGS. 7A and 7B illustrate the height difference, Δh, being abrupt at the junction 706 (e.g., a step increase), the height difference may increase gradually (e.g., from about 0 μm to a maximum height difference). Alternatively, the height difference may decrease gradually (e.g., taper) from a maximum height difference. A gradual increase or decrease in height difference, as used herein, may refer to a continuous incremental increase or decrease in height difference, where an angle between any one differential segment of a height profile and an immediately adjacent differential segment of the height profile is greater than 90°. For example, at the junction 706, a bottom wall of the channel and a bottom wall of the reservoir can meet at an angle greater than 90°. Alternatively or in addition, a top wall (e.g., ceiling) of the channel and a top wall (e.g., ceiling) of the reservoir can meet an angle greater than 90°. A gradual increase or decrease may be linear or non-linear (e.g., exponential, sinusoidal, etc.). Alternatively or in addition, the height difference may variably increase and/or decrease linearly or non-linearly. While FIGS. 7A and 7B illustrate the expanding reservoir cross-section height as linear (e.g., constant expansion angle, (3), the cross-section height may expand non-linearly. For example, the reservoir may be defined at least partially by a dome-like (e.g., hemispherical) shape having variable expansion angles. The cross-section height may expand in any shape.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

Single Cell Assays for Evaluating Transposase Accessible Chromatin Using Sequencing (scATAC-Seq).

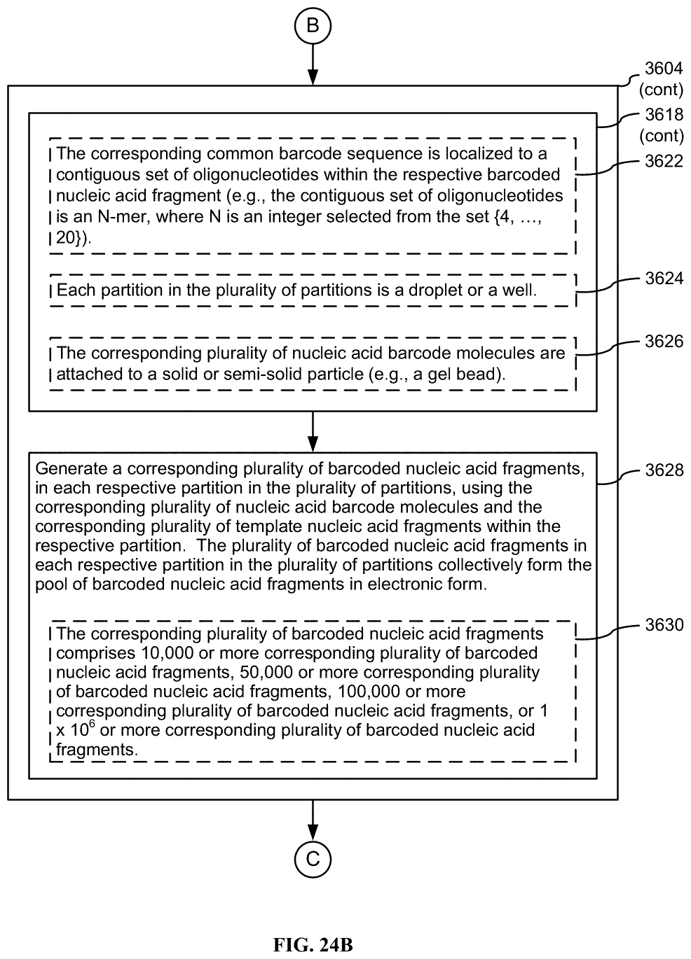

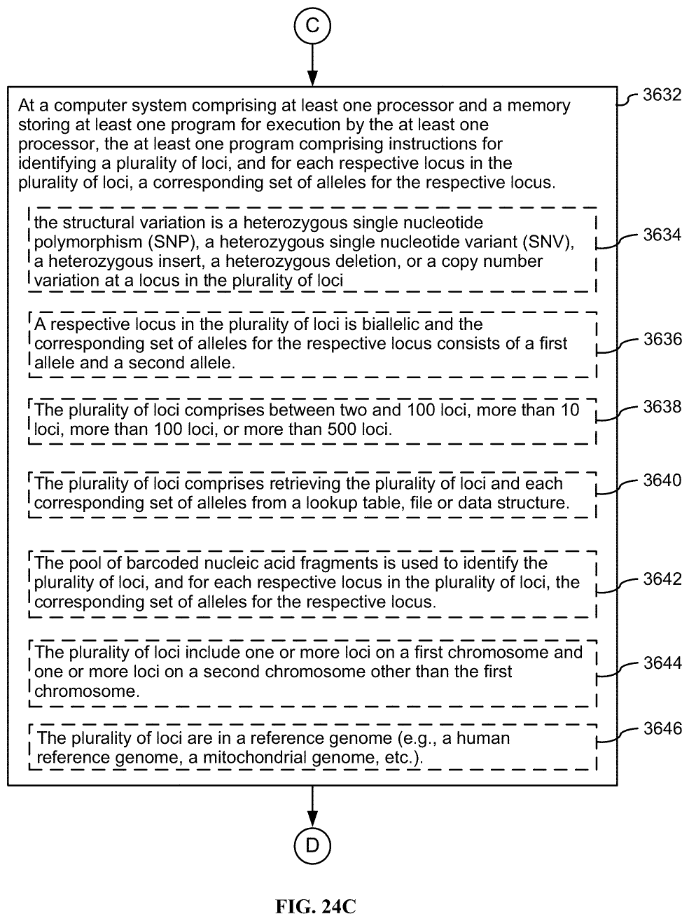

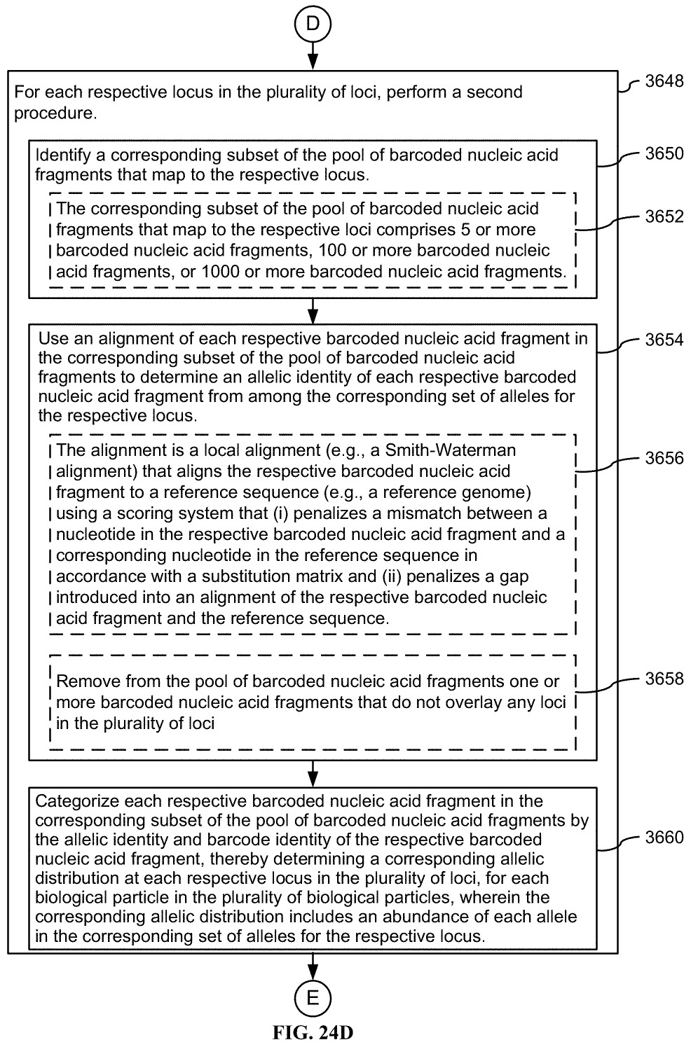

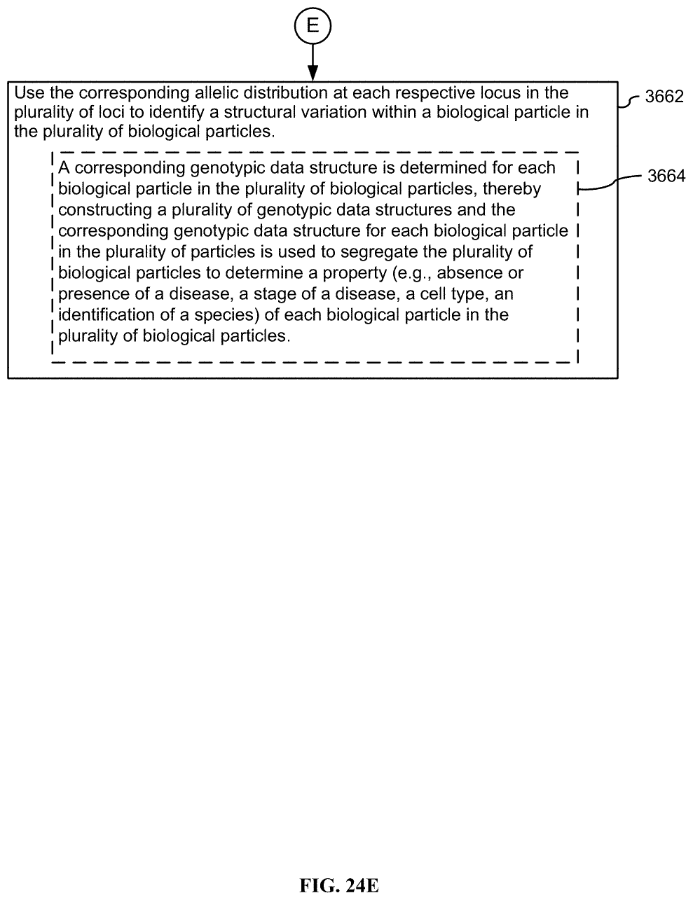

With reference to FIG. 24A, disclosed herein, in some embodiments, are methods for nucleic acid processing. For instance, some embodiments of the present disclosure provide a method for identifying a structural variation in nucleic acids obtained from a biological sample of a subject or identifying transposable accessible chromatin (3602).

In some such approaches, a pool of barcoded nucleic acid fragments is obtained (e.g., in electronic form) from a biological sample (3604). The pool of barcoded nucleic acid fragments comprises barcoded nucleic acid fragments of each locus in a plurality of loci. Each respective barcoded nucleic acid fragment in the pool of barcoded nucleic acid fragments is barcoded with a corresponding barcode in a plurality of barcodes that associates with the respective barcoded nucleic acid fragment within a single corresponding cell in the biological sample.

Such barcoded nucleic acid fragments can be obtained by a number of methods. Generally, such methods call for a pool of barcoded nucleic acid fragments (3604). In each respective biological particle of a plurality of biological particles obtained from a biological sample, a corresponding plurality of template nucleic acid fragments is generated using a transposase-nucleic acid complex comprising a transposase molecule and a transposon end nucleic acid molecule in the respective biological particle (3606). In some embodiments, each biological particle in the plurality of biological particles is a single cell nuclei harvested from its cell (3608). In some embodiments, each biological particle in the plurality of biological particles is a single cell (3610). In some embodiments, the transposase molecule is a native Tn5 transposase, a mutated hyperactive Tn5 transposase, or a Mu transposase (3612). In some embodiments, the transposon end nucleic acid molecule is a Tn5 or modified Tn5 transposon end sequence (3614). In some embodiments, the biological sample is from a single subject or from a plurality of subjects 3616). The concurrent use of a sample from a plurality of subjects is advantageous in some instance to reduce reagent costs. Data from the pooled sample can be resolved using barcodes using the disclosed methods.

In some embodiments, to generate the pool of barcoded nucleic acid fragments, each respective partition (e.g., a droplet or well) in a plurality of partitions is formed that comprises: (a) a respective single biological particle (e.g., a cell or nucleus), (b) a corresponding plurality of template nucleic fragments, and (c) a corresponding plurality of nucleic acid barcode molecules comprising a corresponding common barcode sequent that is unique to the respective single biological particle (3618). In some embodiments, the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×1012} (3620). In some embodiments, the corresponding common barcode sequence is localized to a contiguous set of oligonucleotides within the respective barcoded nucleic acid fragment (e.g., the contiguous set of oligonucleotides is an N-mer, where N is an integer selected from the set {4, . . . , 20}).

In some embodiments, a plurality of template nucleic acid fragments is generated with the aid of a transposase-nucleic acid complex comprising a transposase molecule of the plurality of transposase molecules and a transposon end oligonucleotide molecule of the plurality of transposon end oligonucleotide molecules. A barcoded nucleic acid fragment is then generated using a nucleic acid barcode molecule of the plurality of nucleic acid barcode molecules and a template nucleic acid fragment of the plurality of template nucleic acid fragments.

As another example, a method of generating barcoded nucleic acid fragments is provided that comprises providing: (i) a plurality of biological particles (e.g., cells or nuclei), an individual biological particle of the plurality of biological particles comprising chromatin comprising a template nucleic acid; (ii) a plurality of transposon end nucleic acid molecules comprising a transposon end sequence; and (iii) a plurality of transposase molecules. A plurality of template nucleic acid fragments is then generated in a biological particle of the plurality of biological particles with the aid of a transposase-nucleic acid complex comprising a transposase molecule of the plurality of transposase molecules and a transposon end nucleic acid molecule of the plurality of transposon end nucleic acid molecules. A partition is generated that comprises the biological particle comprising the plurality of template nucleic acid fragments and a plurality of nucleic acid barcode molecules comprising a common barcode sequence. A barcoded nucleic acid fragment is generated using a nucleic acid barcode molecule of the plurality of nucleic acid barcode molecules and a template nucleic acid fragment of the plurality of template nucleic acid fragments.

In some embodiments, the transposase is a Tn5 transposase. In some embodiments, the transposase is a mutated, hyperactive Tn5 transposase. In some embodiments, the transposase is a Mu transposase. In some embodiments, the partitions further comprise cell lysis reagents and/or reagent and buffers necessary to carry out one or more reactions.

In some embodiments, a partition (e.g., a droplet or well) comprises a single cell and is processed according to the methods described herein. In some embodiments, a partition comprises a single cell and/or a single nucleus. The single cell and/or the single nucleus may be partitioned and processed according to the methods described herein. In some cases, the single nucleus may be a component of a cell. In some embodiments, a partition comprises chromatin from a single cell or single nucleus (e.g., a single chromosome or other portion of the genome) and is partitioned and processed according to the methods described herein. In some embodiments, the transposition reactions and methods described herein are performed in bulk and biological particles (e.g., nuclei/cells/chromatin from single cells) are then partitioned such that a plurality of partitions is singly occupied by a biological particle (e.g., a cell, cell nucleus, chromatin, or cell bead). For example, a plurality of biological particles may be partitioned into a plurality of partitions such that partitions of the plurality of partitions comprise a single biological particle. In some embodiments, each partition is a droplet or a well (3624).

In some embodiments, the oligonucleotides described herein comprise a transposon end sequence (transposon end nucleic acid molecule). In some embodiments, the transposon end sequence is a Tn5 or modified Tn5 transposon end sequence. In some embodiments, the transposon end sequence is a Mu transposon end sequence. In some embodiments, the transposon end sequence has a sequence of: AGATGTGTATAAGAGACA (SEQ ID NO: 1).

In some embodiments, the oligonucleotides described herein comprise an R1 sequencing priming region. In some embodiments, the R1 sequencing primer region has a sequence of TCTACACTCTTTCCCTACACGACGCTCTTCCGATCT (SEQ ID NO: 2), or some portion thereof. In some embodiments, the R1 sequencing primer region has a sequence of TCGTCGGCAGCGTCAGATGTGTATAAGAGACAG (SEQ ID NO: 3), or some portion thereof. In some embodiments, the oligonucleotides described herein comprise a partial R1 sequence. In some embodiments, the partial R1 sequence is ACTACACGACGCTCTTCCGATCT (SEQ ID NO: 4).

In some embodiments, the oligonucleotides described herein comprise an R2 sequencing priming region. In some embodiments, the R2 sequencing primer region has a sequence of GTGACTGGAGTTCAGACGTGTGCTCTTCCGATCT (SEQ ID NO: 5), or some portion thereof. In some embodiments, the R2 sequencing primer region has a sequence of GTCTCGTGGGCTCGGAGATGTGTATAAGAGACAG (SEQ ID NO: 6), or some portion thereof.

In some embodiments, the oligonucleotides described herein comprise a T7 promoter sequence. In some embodiments, the T7 promoter sequence is TAATACGACTCACTATAG (SEQ ID NO: 7).

In some embodiments, the oligonucleotides described herein comprise a region at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identity to any one of SEQ ID NO: 1-7.

In some embodiments, the oligonucleotides described herein comprise a P5 sequence. In some embodiments, the oligonucleotides described herein comprise a P7 sequence. In some embodiments, the oligonucleotides described herein comprise a sample index sequence.

In some embodiments, the oligonucleotides described herein (corresponding plurality of nucleic acid barcode molecules of a corresponding particle) are attached to a solid support (e.g., a solid or semi-solid particle such as a bead) (3626). In some embodiments, the oligonucleotides (corresponding plurality of nucleic acid barcode molecules of a corresponding particle) described herein are attached to a bead. In some embodiments, the bead is a gel bead. In some embodiments, the oligonucleotides described herein (corresponding plurality of nucleic acid barcode molecules of a corresponding particle) are releasably attached to a bead (e.g., a gel bead). In some embodiments, the oligonucleotides (corresponding plurality of nucleic acid barcode molecules of a corresponding particle) described herein are single-stranded and the first strand is attached to a bead. In some embodiments, the oligonucleotides (corresponding plurality of nucleic acid barcode molecules of a corresponding particle) described herein are double-stranded or partially double-stranded molecules and the first strand is releasably attached to a bead. In some embodiments, the oligonucleotides (corresponding plurality of nucleic acid barcode molecules of a corresponding particle) described herein are double-stranded or partially double-stranded molecules and the second strand is releasably attached to a bead. In some embodiments, the oligonucleotides (corresponding plurality of nucleic acid barcode molecules of a corresponding particle) described herein are double-stranded or partially double-stranded molecules and both the first and the second strand are releasably attached to a bead or a collection of beads.

In some embodiments, the solid support (e.g., bead, such as a gel bead) comprises a plurality of first oligonucleotides and a plurality of second oligonucleotides. In some embodiments, the first oligonucleotides, the second oligonucleotides, or the combination thereof are releasably attached to a bead. In some embodiments, the first oligonucleotides, the second oligonucleotides, or the combination thereof are double-stranded or partially double-stranded molecules and the first strand is releasably attached to a bead. In some embodiments, the first oligonucleotides, the second oligonucleotides, or the combination thereof are double-stranded or partially double-stranded molecules and the second strand is releasably attached to a bead. In some embodiments, the first oligonucleotides, the second oligonucleotides, or the combination thereof are double-stranded or partially double-stranded molecules and the first strand and the second strand are releasably attached to a bead. In some embodiments, the oligonucleotides, the first oligonucleotides, the second oligonucleotides, or a combination there of are bound to a magnetic particle. In some embodiments, the magnetic particle is embedded in a solid support (e.g., a bead, such as a gel bead).

In some embodiments, the first oligonucleotides couple (e.g., by nucleic acid hybridization) to DNA molecules and the second oligonucleotides couple (e.g., by nucleic acid hybridization) to RNA molecules (e.g., mRNA molecules). In some embodiments, the first oligonucleotide (corresponding plurality of nucleic acid barcode molecules of a corresponding particle) comprises a P5 adaptor sequence, a barcode sequence, and an R1 sequence or partial R1 primer sequence. In some embodiments, the second oligonucleotide (corresponding plurality of nucleic acid barcode molecules of a corresponding particle) comprises a R1 sequence or partial R1 primer sequence, a barcode sequence, a unique molecular identifier (UMI) sequence, and a poly(dT) sequence. In some embodiments, the second oligonucleotide comprises a R1 sequence or partial R1 primer sequence, a barcode sequence, a unique molecular identifier (UMI) sequence, and a switch oligo.

In some embodiments, the first oligonucleotide (corresponding plurality of nucleic acid barcode molecules of a corresponding particle) comprises a P5 adaptor sequence, a barcode sequence, and an R1 sequence or partial R1 primer sequence and is partially double-stranded, and the second oligonucleotide (corresponding plurality of nucleic acid barcode molecules of a corresponding particle) comprises a R1 or partial R1 primer sequence, a barcode sequence, a unique molecular identifier (UMI) sequence, and a poly(dT) sequence. In some embodiments, the first oligonucleotide comprises a P5 adaptor sequence, a barcode sequence, and an R1 or partial R1 primer sequence and is single-stranded, and the second oligonucleotide comprises a R1 or partial R1 sequence, a barcode sequence, a unique molecular identifier (UMI) sequence, and a template switching oligo sequence.

As such, the disclosed methods generate a corresponding plurality of barcoded nucleic acid fragments, in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form (3628). In some embodiments, the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments (3630).

Detection of Nucleic Acid Variants from Sequencing Analysis.

In some cases, methods for nucleic acid processing, such as ATAC-seq, may be used to detect nucleic acid variants. Sequencing data may be obtained from ATAC-seq and subsequent analysis (e.g., single cell ATAC-seq), as described above, and the sequencing data may be analyzed to detect nucleic acid variants. Such detection, in some embodiments, makes use of a plurality of loci, and for each respective locus in the plurality of loci, a corresponding set of alleles for the respective locus. Nucleic acid variants that may be detected include, for example, insertions, deletions, substitutions, structural variants (e.g., chromosomal rearrangement), single nucleotide variants (SNVs), single nucleotide polymorphisms (SNPs), and copy number variations (3634). Identification of nucleic acid variants may be useful, for example, in distinguishing populations and/or species, lineage tracing, etc. In some embodiments, a respective locus in the plurality of loci is biallelic and the corresponding set of alleles for the respective locus consists of a first allele (e.g., wild type or reference allele) and a second (alternative) allele (3636). In some embodiments, the plurality of loci comprises between two and 100 loci, more than 10 loci, more than 100 loci, or more than 500 loci (3640).

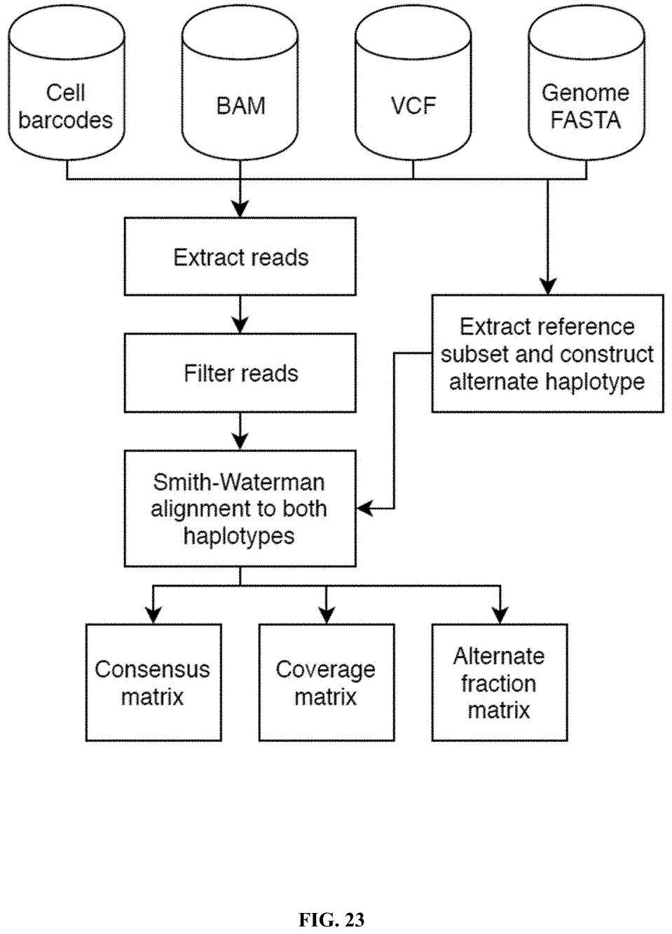

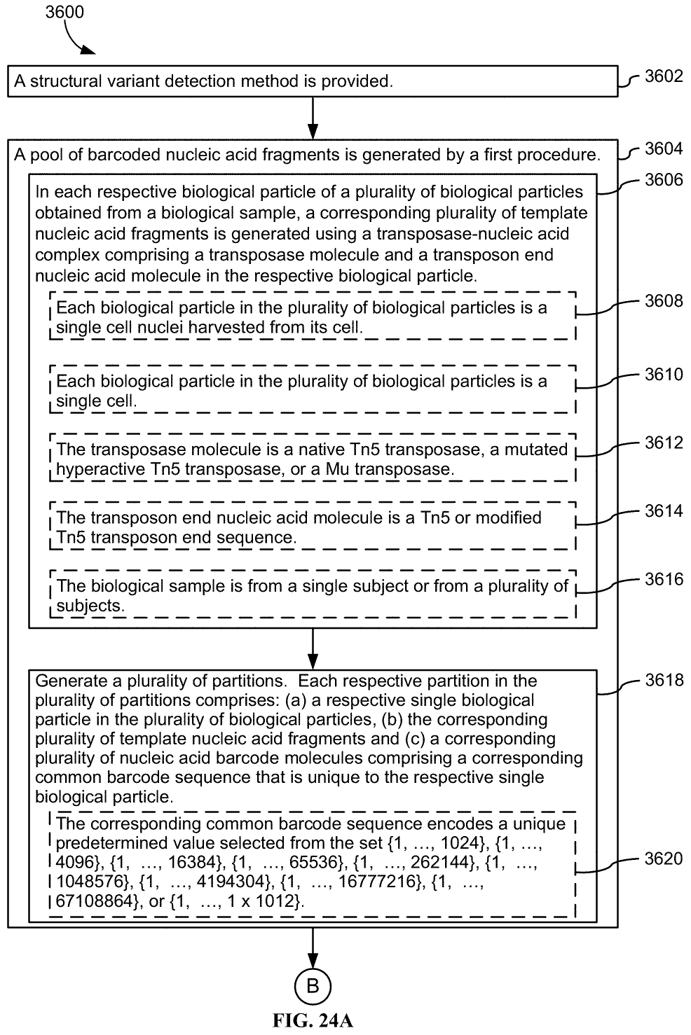

In some embodiments, the plurality of loci comprises retrieving the plurality of loci and each corresponding set of alleles from a lookup table, file or data structure (3640). For instance, in some embodiments, the set of alleles are known variants within the human genome. See, for example, MacDonald et al., “The database of genomic variants: a curated collection of structural variation in the human genome,” Nucleic Acids Res. 2013 Oct. 29. PubMed PMID: 24174537, which is hereby incorporated by reference. In some embodiments, the sequence reads are provided to the program by an electronic data file in a BAM file format, represented in an exemplary workflow in FIG. 23. The BAM file provides inputs to the method comprising sequence reads mapped to the genome, such that the corresponding loci are known a priori.

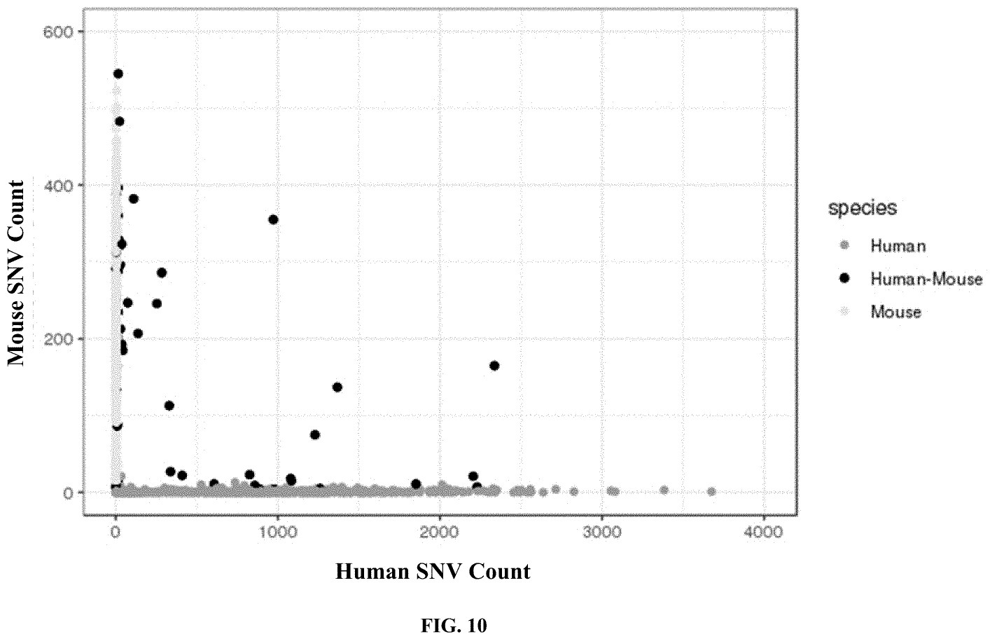

In alternative embodiments, the pool of barcoded nucleic acid fragments is used to identify the plurality of loci, and for each respective locus in the plurality of loci, the corresponding set of alleles for the respective locus (3642). See, for example, FIG. 10 and the description of FIG. 10 below. In some such embodiments, the plurality of loci include one or more loci on a first chromosome and one or more loci on a second chromosome other than the first chromosome (3644). In some such embodiments, the plurality of loci include one or more loci on each of two or more chromosomes, three or more chromosomes, four or more chromosomes, five or more chromosomes, or all the autosomal chromosomes.

In some embodiments, the plurality of loci are in a reference genome (e.g., a human reference genome, a mitochondrial genome, etc.) (3646).

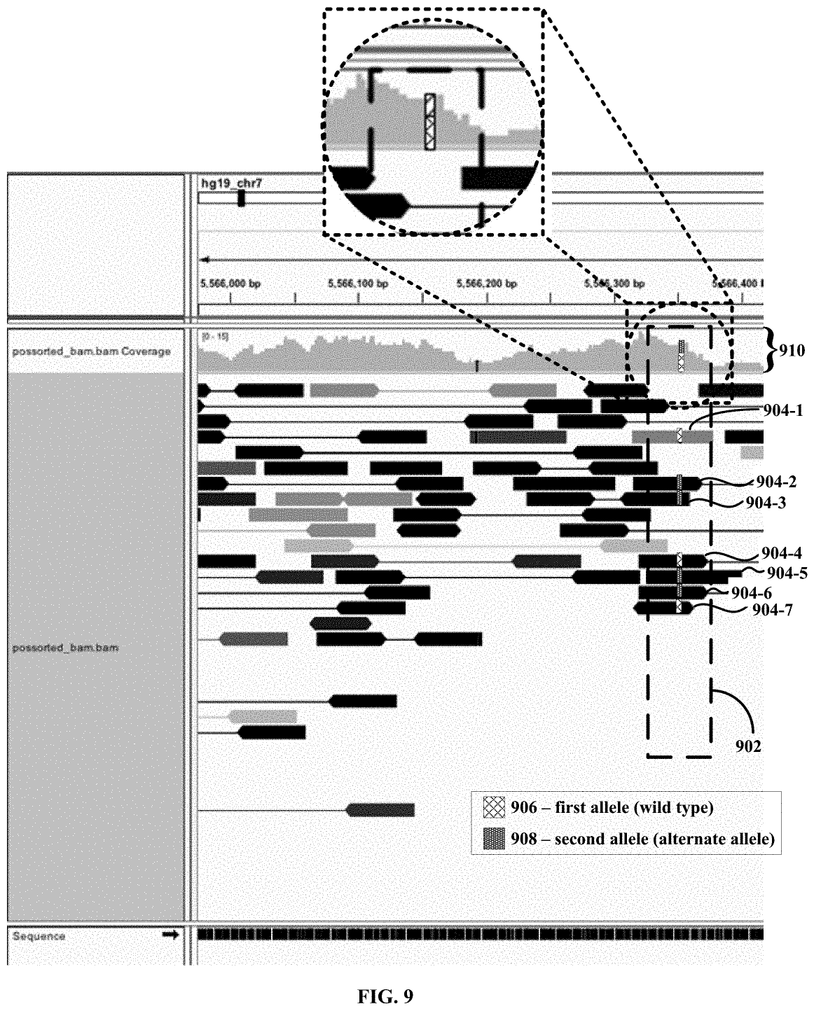

For example, FIG. 9 shows the results from a single cell sequencing experiment, in which a single nucleotide polymorphism (SNPs) is detected in the ACTB gene from human GM12878 cells. To construct the data shown in FIG. 9, a pool of barcoded nucleic acid fragments was generated by a first procedure that comprises generating, in each respective biological particle of a plurality of biological particles obtained from a biological sample of a subject (here, human GM12878 cells), a corresponding plurality of template nucleic acid fragments using a transposase-nucleic acid complex comprising a transposase molecule and a transposon end nucleic acid molecule in the respective biological particle. The first procedure further comprised generating a plurality of partitions. Each respective partition in the plurality of partitions comprised: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. The first procedure further comprised generating a corresponding plurality of barcoded nucleic acid fragments, in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. One or more loci were identified (e.g., here, illustrated in FIG. 9, a particular locus within the ACTB gene is denoted by dashed box 902), and for each respective loci in the one or more loci, a corresponding set of alleles for the respective locus are identified (3632).

Referring to element 3648 of FIG. 24D, for each respective locus in the one or more loci, a second procedure was performed. It comprises identifying a corresponding subset of the pool of barcoded nucleic acid fragments that map to the respective locus (3650). For instance, in FIG. 9 the corresponding subset of the pool of barcoded nucleic acid fragments 904 that extend through box 902 are identified. In some embodiments, the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments (3652).

An alignment of each respective barcoded nucleic acid fragment 904 in the corresponding subset of the pool of barcoded nucleic acid fragments was performed to determine an allelic identity of each respective barcoded nucleic acid fragment from among the corresponding set of alleles for the respective locus (3654). For instance, in FIG. 9, there is a first allele that is wild type, and a second allele that is the alternate allele. Thus, for the locus of box 902, each respective barcoded nucleic acid fragment was identified as having the first allele (wild type) 906 or the second allele (alternate allele) 908.

In some embodiments, a reference sequence (that portion of the genome that the barcoded nucleic acid fragment 904 is aligned to in order to determine the allele of the sequence read at a given genomic position) is all or a portion of a reference genome. In typical embodiments, the entire reference genome is not used for the alignment since respective barcoded nucleic acid fragments 904 have already been mapped to a given loci. Thus, in some alternative embodiments, the reference sequence that is used to perform the alignment is a flanking sequence of 50 nucleotides or less from the given loci, 100 nucleotides or less from the given loci, 200 nucleotides or less from the given loci, or 500 nucleotides or less from the given loci, 500 nucleotides or less from the given loci, 1000 nucleotides or less from the given loci, 2000 nucleotides or less from the given loci, 5000 nucleotides or less from the given loci, 10000 nucleotides or less from the given loci, or 100,000 nucleotides or less from the given loci. The amount of flanking region used in the alignment will depend on the type of sequencing that was used to generate the barcoded nucleic acid fragments 904, the average length of such barcoded nucleic acid fragments 904, and/or on the size of the given loci.

In some embodiments a mismatch between a nucleotide in the barcoded nucleic acid fragment 904 and a corresponding nucleotide in the reference sequence is determined using a substitution matrix. In some embodiments, a gap introduced into an alignment of the barcoded nucleic acid fragment 904 and the reference sequence is penalized. Examples where such scoring is used are the local sequence alignment algorithms of Smith-Waterman (see, for example, Smith and Waterman, J Mol. Biol., 147(1):195-97 (1981), which is incorporated herein by reference), Lalign (see, for example, Huang and Miller, Adv. Appl. Math, 12:337-57 (1991), which is incorporated by reference herein), and PatternHunter (see, for example, Ma B. et al., Bioinformatics, 18(3):440-45 (2002), which is incorporated by reference herein).

As such, in some embodiments, an alignment of respective barcoded nucleic acid fragments (that have been mapped) is needed to determine their allele. In principle, one way to accomplish this is to, for each allele in the set of alleles for the respective loci to which a respective barcoded nucleic acid fragment has been mapped, generate a reference sequence in the vicinity of the loci and perform an alignment of the barcoded nucleic acid fragment to each such reference sequence. For instance, in some implementations, consider a given loci to which the respective barcoded nucleic acid fragment has a reference allele and an alternative allele. The respective barcoded nucleic acid fragment is thus aligned to a reference sequence for the reference allele at the given locus. The respective barcoded nucleic acid fragment is also aligned to a reference sequence for the alternative allele at the given locus. The alignment that has the best alignment score is called as the allele, in the set of alleles, for the respective barcoded nucleic acid fragment at the given locus.

In some embodiments, the alignment is a local alignment (e.g., a Smith-Waterman alignment) that aligns the respective barcoded nucleic acid fragment to a reference sequence (e.g., a reference genome) using a scoring system that (i) penalizes a mismatch between a nucleotide in the respective barcoded nucleic acid fragment and a corresponding nucleotide in the reference sequence in accordance with a substitution matrix and (ii) penalizes a gap introduced into an alignment of the respective barcoded nucleic acid fragment and the reference sequence (3656). Examples of programs that can serve to map barcoded nucleic acid fragment to reference sequences include, but are not limited to SARUMAN, GPU-RMAP, BarraCUDA, SOAP3, SOAP3-dp, CUSHAW, CUSHAW2-GPU, Burrows-Wheeler transform algorithm, a hashing algorithm, pigeonhole, MAQ, RMAP, SOAP, Hobbes, ZOOM, FastHASH, RazerS, RazerS 3, BFAST SEME, SHRiMP, BWT-SW, BWA, Botie, BLASR, Bowtie 2, BWA-SW, GEM, or SOAP2. For further discussion of these mapping algorithms, see Canzar and Stazberg, 2018, “Short Read Mapping: An Algorithmic Tour,” Proc IEEE Inst. Electr Electron Eng., 105(3), 436-458, which is hereby incorporated by reference.

In some embodiments, there is removed from the pool of barcoded nucleic acid fragments one or more barcoded nucleic acid fragments that do not overlay any loci in the plurality of loci (3658). The filtering step is represented in the exemplary workflow in FIG. 23.

In the disclosed methods, each respective barcoded nucleic acid fragment in the corresponding subset of the pool of barcoded nucleic acid fragments is categorized by the allelic identity and barcode identity of the respective barcoded nucleic acid fragment, thereby determining a corresponding allelic distribution at each respective locus in the plurality of loci, for each biological particle in the plurality of biological particles, where the corresponding allelic distribution includes an abundance of each allele in the corresponding set of alleles for the respective locus (3660). For instance, in some embodiment, each respective barcoded nucleic acid fragment 904 in the corresponding subset of the pool of barcoded nucleic acid fragments is identified by the allelic identity of the respective barcoded nucleic acid fragment and the barcode identity of the respective barcoded nucleic acid fragment which tracks it back to a particular biological fragment, thereby determining a corresponding allelic distribution at each respective locus in the plurality of loci, for each biological particle in the plurality of biological particles. The corresponding allelic distribution includes an abundance of each allele in the corresponding set of alleles for the respective locus. This is illustrated in graph 910 of FIG. 9, which shows, for each respective position in a range of positions along a reference sequence (e.g., a reference genome), the coverage of the barcoded nucleic acid fragments for the respective position. Thus, for the locus within box 904, the coverage is represented on the Y-axis of graph 910, and the proportion of the barcoded nucleic acid fragments having the first allele that contribute to this coverage is graphically denoted within the graph 910 by the hashing 906 representative of the first allele. Likewise, the proportion of the barcoded nucleic acid fragments having the second allele that contribute to this coverage is graphically denoted within the graph 910 by the hashing 908 representative of the second allele. In this way, the corresponding allelic distribution at each respective locus (e.g., including the locus within box 902) in the plurality of loci is used to identify a structural variation or a copy number variation (3662). For instance, as illustrated in FIG. 9, it is seen that an alternate allele is exhibited by a certain proportion of the barcoded nucleic acid fragments obtained from a biological sample of a subject (human GM12878 cells).

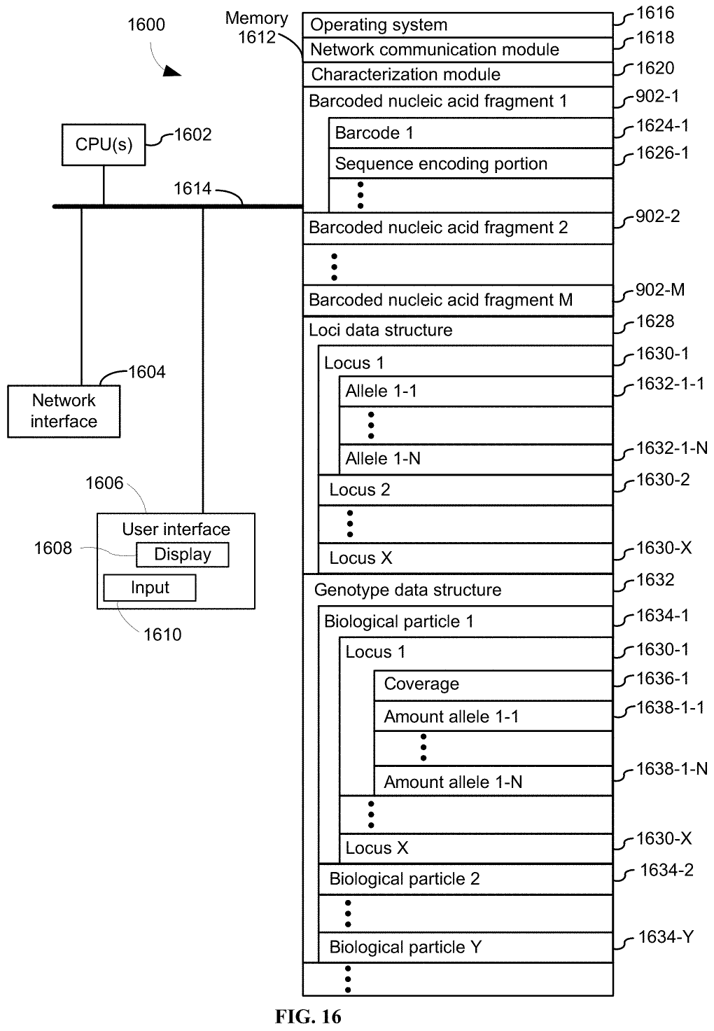

While the disclosed methods can be used to identify a single structural variation (SNV) at a single locus as illustrated in FIG. 9, in some embodiments, the disclosed methods identify a set of SNVs for each biological particle in the plurality of biological particles in a biological sample. As such, as illustrated in FIG. 16, in some embodiments the disclosed methods result in the acquisition of a genotype data structure 1633 that tracks, for each respective biological particle 1634-1, 1634-2, 1634-Y inclusive, in a plurality of biological particles, for each respective locus 1630-1, 1630-2, and 1630-X inclusive, in a plurality of loci: (i) optionally, a coverage of the respective locus 1630 for the respective biological particle 1634 and (ii) a proportion 1638-1-1, . . . , 1638-1-N inclusive of each allele 1632 in the corresponding set of alleles for the respective locus 1630 observed for the biological particle 1634.

The proportions 1638 can be used to call a SNV at a given locus for a given biological particle. For instance, in some embodiments, when there are two alleles at a given locus (e.g., a wild type allele and an alternate allele), the given locus is called as a SNV when the proportion of alternate allele (relative to 100 percent) is greater than 5, 10, 15, 20, 25, 30, 35, 40, 45, 50, 55, 60, 65, 70, 75, 80, 85, 90, 95, 96, 97, 98, or 99 percent. Such SNV calls give rise to a set of SNVs for each biological particle in the plurality of biological particles using the disclosed methods. The set of SNVs for each given biological particle are indicative of the type of biological particle that is present. FIG. 10 illustrates the point. FIG. 10 shows the results from a single cell sequencing experiment, where single nucleotide variants (SNV) are used to distinguish human GM12878 cells from EL4 mouse cells. That is, for FIG. 10, the biological sample that is analyzed in accordance with the disclosed methods is a mixture of human GM12878 cells and mouse EL4 cells. There is a corresponding set of SNVs that have been determined in the manner discussed above. Here, the set of SNVs includes the SNVs that are found in the human GM12878 cells and the SNVs that are found in the mouse EL4 cells. The scATAC-seq data of the GM12878 cells and the EL4 cells is analyzed to derive a count of the number human SNVs and the number of EL4 SNVs that are in each such cell. For this, the barcodes of the barcoded nucleic acid fragments in each respective biological particle are used as validation to ascertain the origin of each respective biological particle (human or mouse). In FIG. 10, each respective biological particle is plotted with Cartesian coordinates X and Y, where X is the human SNV count in the respective biological particle, and Y is the mouse SNV count in the respective particle, and each such plotted particle is lightly shaded if the barcode derived biological identity is mouse, intermediately shaded if the barcode derived biological identity is human, and darkly shaded the barcode derived biological identity indicates that the biological particle is both mouse and human. As illustrated in FIG. 10, the disclosed methods correctly segregate the human cells close to the X-axis and the mouse cells close to the Y-axis. It is believed that those biological particles that are identified as both human and mouse represent doublets in which a single partition acquired both a single mouse cell and a single human cell. FIG. 10 thus illustrates determining a corresponding genotypic data structure 1633 for each biological particle 1634 in the plurality of biological particles, thereby constructing a plurality of genotypic data structures and further using the corresponding genotypic data structure for each biological particle in the plurality of biological particles to segregate the plurality of biological particles to determine a property of each biological particle in the plurality of biological particles. Here, in FIG. 10, the property is the species of origin of each respective biological particles. However, in other embodiments the biological property is absence or presence of a disease (e.g., cancer). In still other embodiments the biological property is a stage of a disease (e.g., a stage of a particular type of cancer). FIG. 10 further illustrates that the set of SNVs of any given cell is quite unique because the cells along the X-axis and the Y-axis do not cluster on top of each other. Rather, each biological particle tends to have a unique number of SNPs.

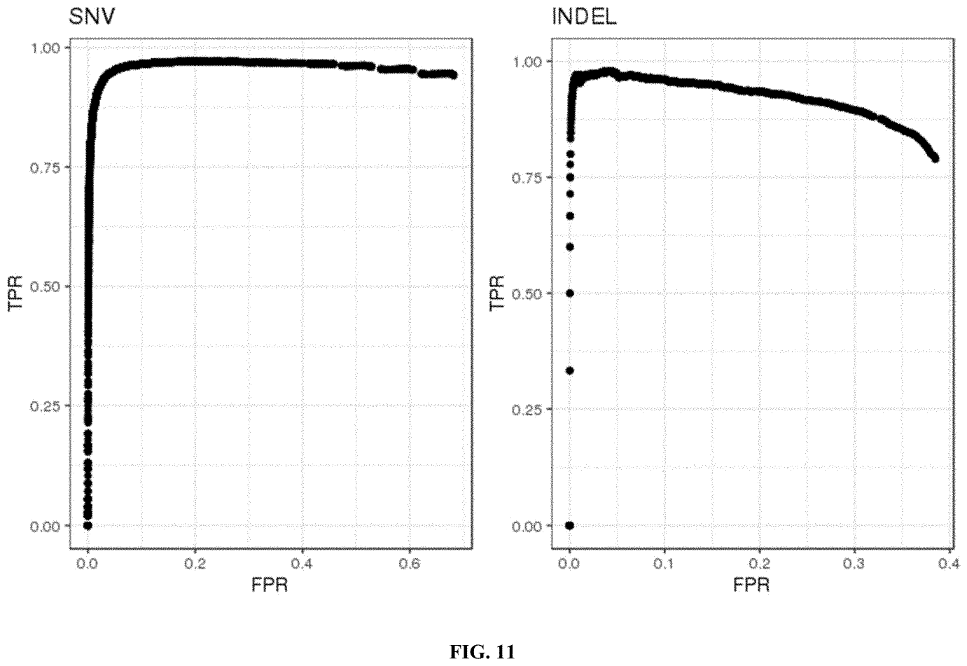

FIG. 11 shows the performance of variant detection in GM12878 cells using the disclosed single cell analysis methods. In each chart in FIG. 11, the X-axis is the False Positive Rate (FPR), and the Y-axis is the True Positive Rate (TPR).

As used herein, the term “sensitivity” or TPR refers to the number of true positives (TP) divided by the sum of the number of true positives and false negatives (FN) across the population of GM12878 cells for a given SNV (left panel of FIG. 11) or INDEL (right panel of FIG. 11). Here, the term “false negative” refers to an actual SNV in the GM12878 reference genome that has not been identified by the disclosed methods in a GM12878 reference cell. Thus, the number of false negatives across the population of GM12878 cells, for a given SNV or INDEL, is the total number of times across the population of GM12878 cells that the disclosed methods failed to identify the SNV or INDEL in the GM12878 cells. For instance, if the disclosed methods failed to identify the SNV or INDEL in two of the GM12878 cells, the total number of times across the population of GM12878 cells that the disclosed methods failed to identify the SNV or INDEL would be two. Here, the term “true positive” refers to an actual SNV in the GM12878 reference genome that has been correctly identified by the disclosed methods in a GM12878 reference cell. Thus, the number of true positives across the population of GM12878 cells, for a given SNV or INDEL, is the total number of times across the population of GM12878 cells that the disclosed methods correctly identified the SNV or INDEL. For instance, if the disclosed methods correctly identified the SNV in two GM12878 cells, the total number of times across the population of GM12898 cells that the disclosed methods correctly identified the SNV or INDEL would be two.

As used herein, the term “false positive rate” or FPR refers to the number of false positives divided by the sum of the number of false positives and true negatives across the population of GM12878 cells for a given SNV (left panel of FIG. 11) or INDEL (right panel of FIG. 11). Here, the term “false positive” refers to a SNV or INDEL identified in a GM12878 cell that in fact does not exist in the GM12878 reference genome. Thus, the number of false positives across the population of GM12878 cells, for a given SNV or INDEL, is the total number of times across the population of GM12878 cells that the disclosed methods identified SNV or INDEL that, in fact, does not exists. For instance, if the disclosed methods identified the SNV or INDEL in two GM12878 reference cells the total number of times across the population of GM12878 cells that the disclosed methods identified the SNV or INDEL would be two. Here, the term “true negative” refers to an absence of a SNV or INDEL in the GM12878 reference genome that has been correctly identified by the disclosed methods in a GM12878 reference cell as being absent. Thus, the number of true negatives across the population of GM12878 cells, for a given SNV or INDEL, is the total number interrogated GM12878 cells that the disclosed methods correctly identified as not having the SNV or INDEL. For instance, if the disclosed methods correctly identified that two of the GM12878 reference cells do not have the SNV or INDEL, the total number of GM12878 reference cells that do not have the SNV or INDEL would be two.

Thus, the left hand panel of FIG. 11 is a plot of the TPR versus the FPR for each SNV assayed in the population of GM12878 cells. The right hand panel of FIG. 11 is a plot of the TPR versus the FPR for each INDEL assayed in the population of GM12878 cells. Thus, in the left hand panel, each point represents a single different SNV in the GM12878 reference genome. In the right hand panel, each point represents a single different INDEL in the GM12878 reference genome. The charts show that, for both SNVs and INDELs as FPR goes up, the TPR is not appreciably dropping off. Thus, FIG. 11 shows that the disclosed systems and method are able to identify SNPs and INDEL with suitable performance.

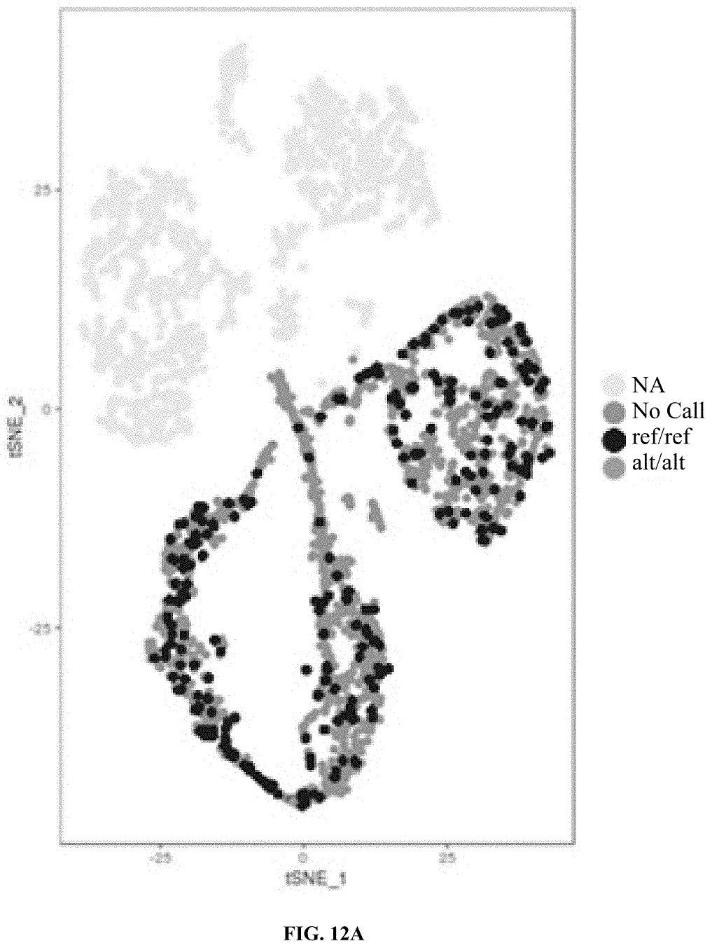

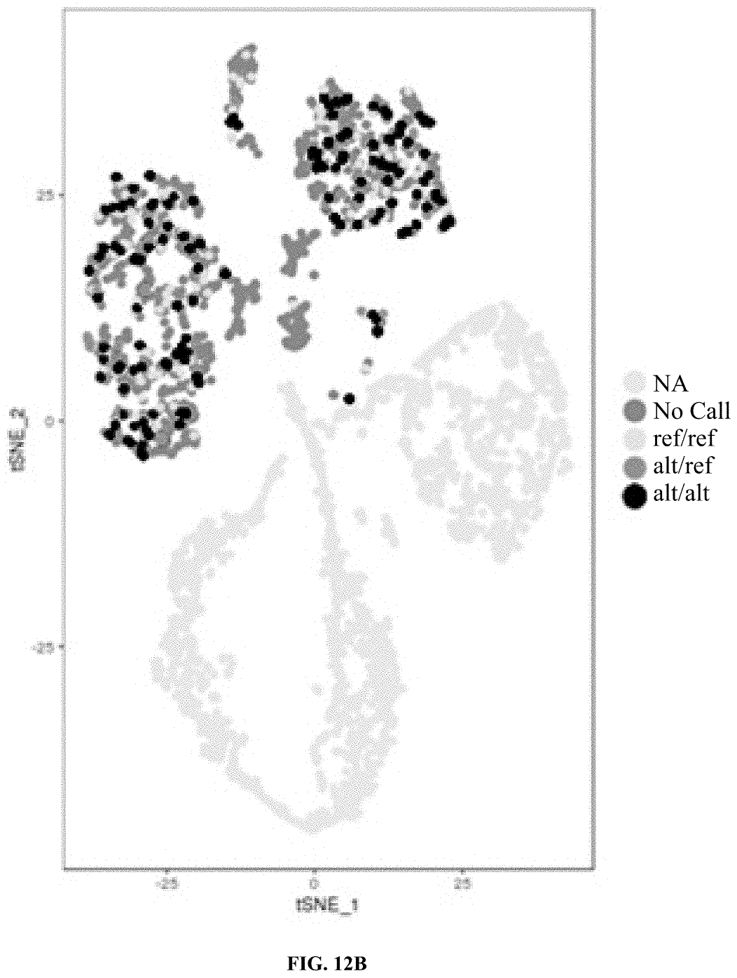

FIGS. 12A and 12B shows the results from a single cell sequencing experiment, where sequences from the peripheral blood mononuclear cells (PBMCs) cells of two subjects (Donor 1 and Donor 2) were mixed and analyzed for SNVs, such that SNVs specific to each individual were identified. FIG. 12A grey scale codes for a particular allelic position (Chr1.564995) that is the site of a SNV specific to Donor 2, and therefore shows how the cells from Donor 2 cluster together in the bottom right portion of the graph. FIG. 12B grey scale codes for a particular allelic position (Chr1.624866) that is the site of a SNV specific to Donor 1, and therefore shows how the cells from Donor 1 cluster together in the upper left hand portion of the graph.

To produce the data shown in FIGS. 12A and 12B, a first pool of barcoded nucleic acid fragments was obtained from Donor 1 by a first procedure that comprises (i) generating, in each respective cell of a first plurality of cells obtained from Donor 1, a corresponding plurality of template nucleic acid fragments using a transposase-nucleic acid complex comprising a transposase molecule and a transposon end nucleic acid molecule in the respective cell. Further, a first plurality of partitions was created, where each respective partition in the first plurality of partitions comprises: (a) a respective single biological particle in the first plurality of cells, (b) the corresponding plurality of template nucleic acid fragments and (c) a corresponding plurality of nucleic acid barcode molecules comprising a corresponding common barcode sequence that is unique to the respective single cell. Further, a corresponding plurality of barcoded nucleic acid fragments was created, in each respective partition in the first plurality of partitions, using the corresponding plurality of nucleic acid barcode molecules and the corresponding plurality of template nucleic acid fragments within the respective partition, where the plurality of barcoded nucleic acid fragments in each respective partition in the first plurality of partitions collectively form the first pool of barcoded nucleic acid fragments in electronic form. The same procedure was performed using the plurality of cells obtained from Donor 2, thereby forming a second pool of barcoded nucleic acid fragments in electronic form derived from Donor 2.

Further, the first pool of barcoded nucleic acid fragments from Donor 1 was used to identify a first plurality of loci using a variant calling procedure (e.g., GATK4 or Mutect), and for each respective locus in the first plurality of loci, a corresponding set of alleles for the respective locus (e.g., using GATK4 or Mutect). This first plurality of loci thus represent the loci that have a SNP within Donor 1 cells.

Further, the second pool of barcoded nucleic acid fragments, from Donor 2, was used to identify a second plurality of loci, and for each respective locus in the second plurality of loci, a corresponding set of alleles for the respective locus (e.g., using GATK4 or Mutect). This second plurality of loci thus represent the loci that have a SNP within Donor 2 cells. The second plurality of loci are thus different than the first plurality of loci.

For each respective locus in both the first and second plurality of loci, a second procedure was performed that comprised i) identifying a corresponding subset of the first pool of barcoded nucleic acid fragments that map to the respective locus, ii) using an alignment of each respective barcoded nucleic acid fragment in the corresponding subset of the first pool of barcoded nucleic acid fragments to determine an allelic identity of each respective barcoded nucleic acid fragment from among the corresponding set of alleles for the respective locus, and iii) categorizing each respective barcoded nucleic acid fragment in the corresponding subset of the pool of barcoded nucleic acid fragments by the allelic identity of the respective barcoded nucleic acid fragment. Referring to FIG. 16, a corresponding genotypic data structure 1633 was constructed for each cell (biological particle) 1634 in the first plurality of cells (from Donor 1) using the barcode identities of the first pool of barcoded nucleic acid fragments and the identified alleles for each such barcoded nucleic acid fragment (from among the first and second plurality of loci) thereby constructing a first plurality of genotypic data structures, one for each cell in the first plurality of cells.

For each respective locus in both the first and second plurality of loci, a third procedure was performed that comprised i) identifying a corresponding subset of the second pool of barcoded nucleic acid fragments that map to the respective locus, ii) using an alignment of each respective barcoded nucleic acid fragment in the corresponding subset of the second pool of barcoded nucleic acid fragments to determine an allelic identity of each respective barcoded nucleic acid fragment from among the corresponding set of alleles for the respective locus, and iii) categorizing each respective barcoded nucleic acid fragment in the corresponding subset of the pool of barcoded nucleic acid fragments by the allelic identity of the respective barcoded nucleic acid fragment (from among the first and second plurality of loci). Referring to FIG. 16, a corresponding genotypic data structure 1633 was constructed for each cell 1634 in the second plurality of cells (from Donor 2) using the barcode identities of the second pool of barcoded nucleic acid fragments and the identified alleles for each such barcoded nucleic acid fragment thereby constructing a second plurality of genotypic data structures, one for each cell in the second plurality of cells.

The corresponding genotypic data structure for each cell in the first and second plurality of cells was used to segregate the cells to determine a property of each cell. Referring to FIGS. 12A and 12B, for each respective cell, a corresponding vector was generated whose elements comprised the allelic identity of the respective cell for each locus in the first and second plurality of loci. Referring to FIG. 16, one manner in which this is done is to call, for each locus 1630, the corresponding allele 1638 that was observed most often in the barcoded nucleic acid fragments attributed, through barcoding, to the respective cell. These vectors therefore represent the alleles of each respective cell in the first and second plurality of cells for each locus in the first and second plurality of loci in multidimensional space. These vectors were compressed to two dimensional space using t-distributed stochastic neighboring entities (t-SNE). The t-SNE algorithm is a machine learning algorithm for dimensionality reduction. See van der Maaten and Hinton, 2008, “Visualizing High-Dimensional Data Using t-SNE,” Journal of Machine Learning Research 9, 2579-2605, which is hereby incorporated by reference. The nonlinear dimensionality reduction technique t-SNE is particularly well-suited for embedding high-dimensional data (here, the allelic values for each locus in the first and second plurality of loci) computed for each respective cell in the first and second plurality of cells based upon the measured discrete attribute value (e.g., allele value) of each locus in a respective cell into a space of two dimensions, which can then be visualized as a two-dimensional visualization (e.g. the t-SNE plot of FIGS. 12A and 12B). As such, t-SNE uses the corresponding genotypic data structure for each cell in the first and second plurality of cells to segregate the plurality of cells into two clusters, one of which represents Donor 1, and the other of which represents Donor 2. As such, the disclosed methods are able to use the SNV values from the scATAC-seq data to determine a property of the cells: whether they originate from Donor 1 or Donor 2. It will be appreciated that such a technique can be used as the basis for training a classifier that discriminates between two biological states such as absence or presence of a particular disease (e.g., cancer), or stage of a disease (e.g., a stage of cancer).

Turning to FIG. 12A, the above described t-SNE process provides a “dot” for each respective vector. Each respective vector corresponds to the allelic calls across the first and second plurality of alleles for a corresponding cell. Thus, each “dot” in FIG. 12A represents a cell in the first (Donor 1) or second (Donor 2) plurality of cells. Next, each “dot” in FIG. 12A is grey-scale coded by the allelic call made for the corresponding cell at genomic position chr 1:564995, a genomic location that the variant calling described above identified as possessing an SNV in Donor 2. It is seen from FIG. 12A that the biological particles from Donor 2 that identify genomic position chr 1:564995 with either the ref/ref or alt/alt allelic value for genomic position chr 1:564995 cluster together in the bottom right portion of the graph. Turning to FIG. 12B, the same above described t-SNE plot of FIG. 12A is provided in which there is a “dot” for each respective vector. Each respective vector corresponds to the allelic calls across the first and second plurality of alleles for a corresponding cell. Thus, each “dot” in FIG. 12B again represents a cell in the first or second plurality of cells. Next, each “dot” in FIG. 12B is grey-scale coded by the allelic call made for the corresponding cell at genomic position chr 1:1624866, a genomic location that the variant calling described above identified as possessing an SNV in Donor 1. It is seen from FIG. 12B that the cells from Donor 1 that identify genomic position chr 1:1624866 with either the ref/ref, alt/ref, or alt/alt allelic value for genomic position chr 1:1624866 cluster together in the upper left portion of the graph. Thus, FIGS. 12A and 12B show that the two clusters formed by t-SNE in fact represent the segregation of Donor 1 and Donor 2 cells on the basis of the genotypic data structures constructed from the above-disclosed processing of scATAC-seq data. Thus, shows FIGS. 12A and 12B shows how a corresponding genotypic data structure is determined for each biological particle in a plurality of biological particles, thereby constructing a plurality of genotypic data structures and how the corresponding genotypic data structure for each biological particle in the plurality of particles is used to segregate the plurality of biological particles to determine a property (e.g., absence or presence of a disease, a stage of a disease, a cell type, an identification of a species) of each biological particle in the plurality of biological particles (3664).

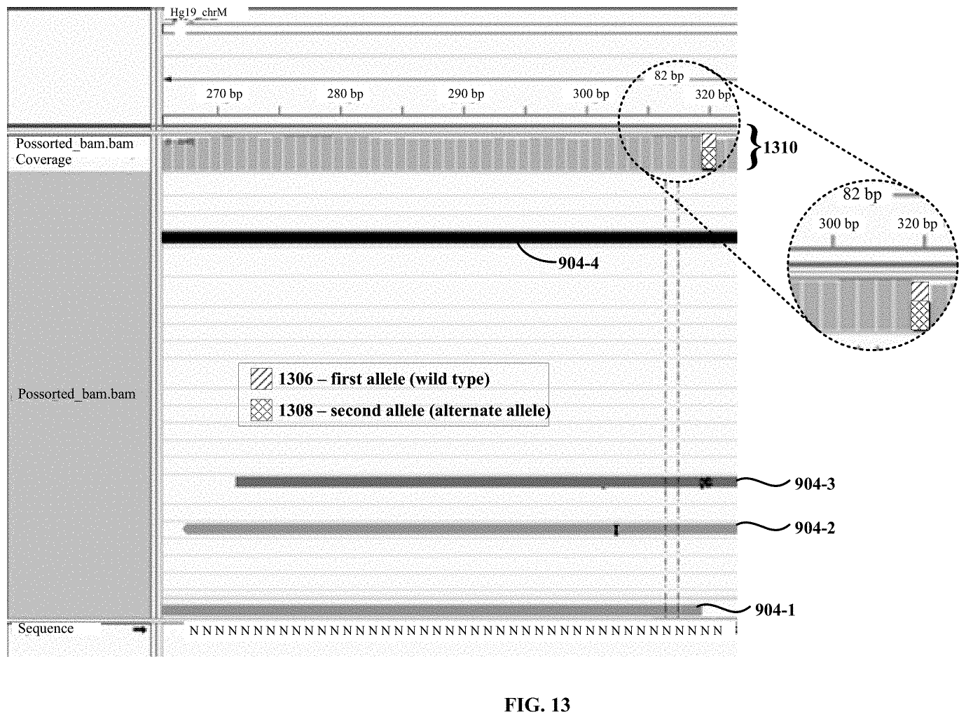

FIG. 13 shows an example of detecting a SNP in a human mitochondria region from single cell sequencing data obtained from human GM12878 cells. For FIG. 13, a pool of barcoded nucleic acid fragments is generated by a first procedure that comprises generating, in each respective biological particle of a plurality of biological particles obtained from a biological sample of a subject (here, human GM12878 nuclei and associated mitochondrial nucleic acid), a corresponding plurality of template nucleic acid fragments using a transposase-nucleic acid complex comprising a transposase molecule and a transposon end nucleic acid molecule in the respective biological particle. The first procedure further comprises generating a plurality of partitions. Each respective partition in the plurality of partitions comprises: (a) a respective single biological particle (here, human GM12878 nuclei and associated mitochondrial nucleic acid) in the plurality of biological particles, (b) the corresponding plurality of template nucleic acid fragments and (c) a corresponding plurality of nucleic acid barcode molecules comprising a corresponding common barcode sequence that is unique to the respective single biological particle. The first procedure further comprises generating a corresponding plurality of barcoded nucleic acid fragments, in each respective partition in the plurality of partitions, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. In this way, a substantial portion of the barcoded nucleic acid fragments are mitochondrial and exhibit single cell behavior (in that they have been barcoded on a single cell basis). One or more loci are identified (e.g., here, in FIG. 13, a particular locus within the human mitochondrial genome), and for each respective loci in the one or more loci, a corresponding set of alleles for the respective locus are identified.

For each respective locus in the one or more loci, a second procedure is performed that comprises i) identifying a corresponding subset of the pool of barcoded nucleic acid fragments that map to the respective locus. For instance, in FIG. 13 the corresponding subset of the pool of barcoded nucleic acid fragments 904 that map to the locus of interest in FIG. 13 are identified. An alignment of each respective barcoded nucleic acid fragment 904 in the corresponding subset of the pool of barcoded nucleic acid fragments is performed to determine an allelic identity of each respective barcoded nucleic acid fragment from among the corresponding set of alleles for the respective locus. For instance, in FIG. 13, there is a first allele that is wild type, and a second allele that is the alternate allele. Thus, for the locus of FIG. 13, each respective barcoded nucleic acid fragment is identified as having the first allele (wild type) 1306 or the second allele (alternate allele) 1308. Each respective barcoded nucleic acid fragment 904 in the corresponding subset of the pool of barcoded nucleic acid fragments is identified by the allelic identity of the respective barcoded nucleic acid fragment and the barcode identity of the respective barcoded nucleic acid fragment that tracks it back to a particular biological fragment, thereby determining a corresponding allelic distribution at each respective locus in the plurality of loci, for each cell in the plurality of cells. The corresponding allelic distribution includes an abundance of each allele in the corresponding set of alleles for the respective locus. This is illustrated in graph 1310 of FIG. 13, which shows, for each respective position in a range of positions along a reference sequence (e.g., a reference genome), the coverage of the barcoded nucleic acid fragments for the respective position. Thus, for the locus of interest in FIG. 13, the coverage is represented on the Y-axis of graph 1310, and the proportion of the barcoded nucleic acid fragments having the first allele that contribute to this coverage is graphically denoted within the graph 1310 by the hashing 1306 representative of the first allele. Likewise, the proportion of the barcoded nucleic acid fragments having the second allele that contribute to this coverage is graphically denoted within the graph 1310 by the hashing 1308 representative of the second allele. In this way, the corresponding allelic distribution at each respective locus (e.g., including the locus of interest in FIG. 13) in the plurality of loci is used to identify a structural variation or within the mitochrondrial DNA. For instance, as illustrated in FIG. 13, it is seen that an alternate allele is exhibited by a certain proportion of the barcoded nucleic acid fragments obtained from a biological sample of a subject (human GM12878 cells).

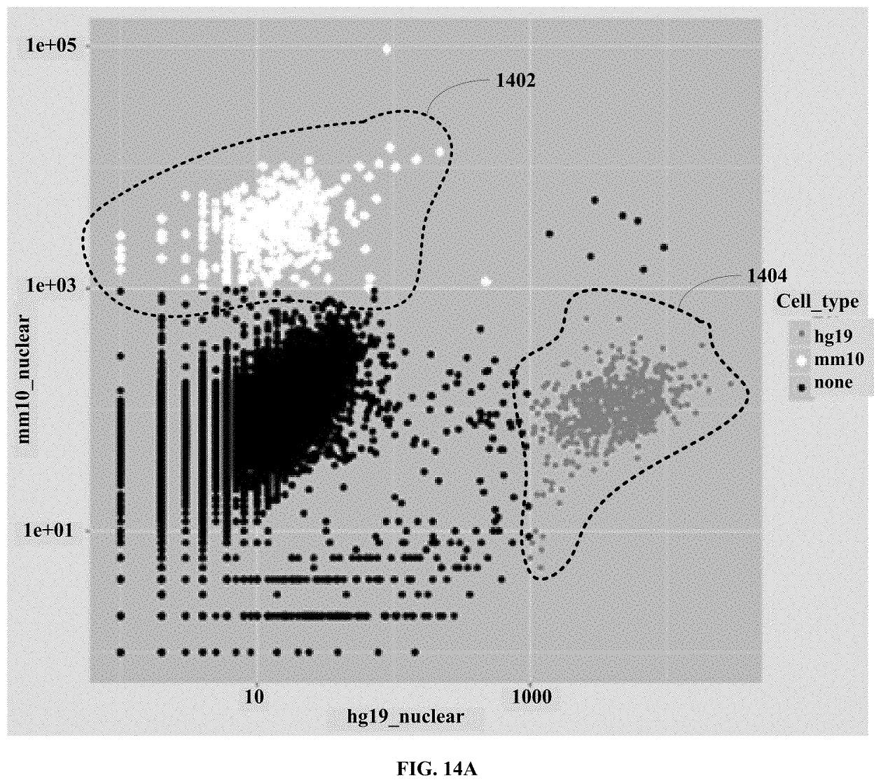

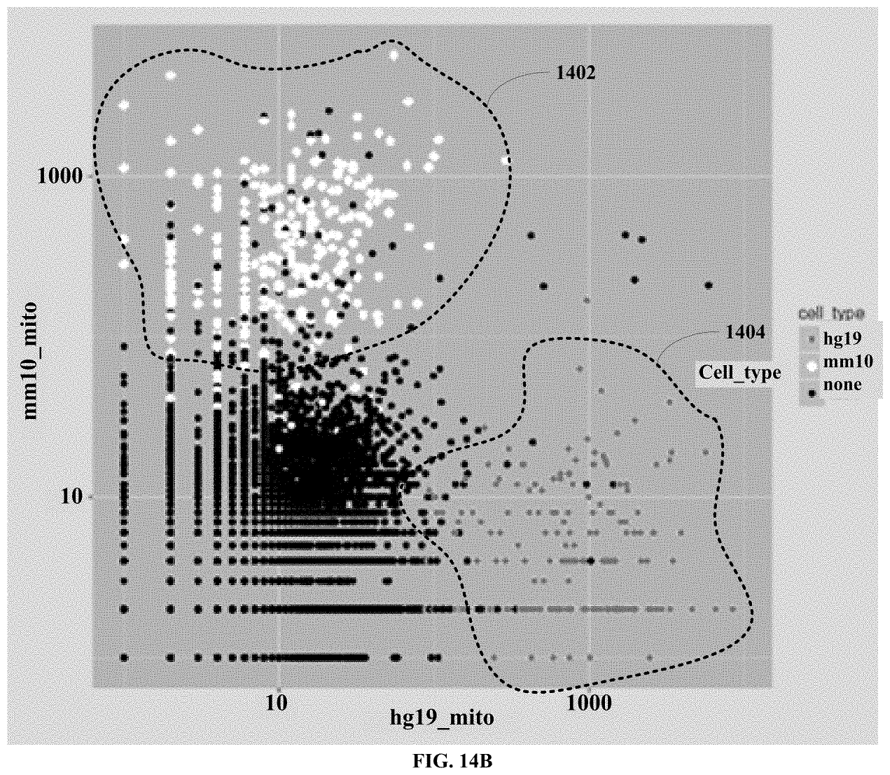

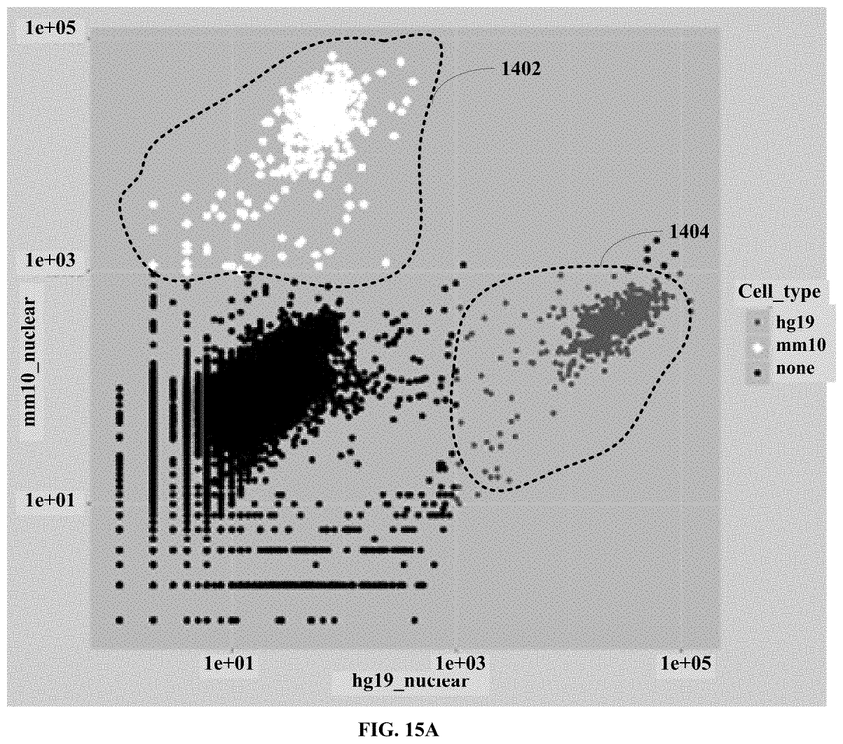

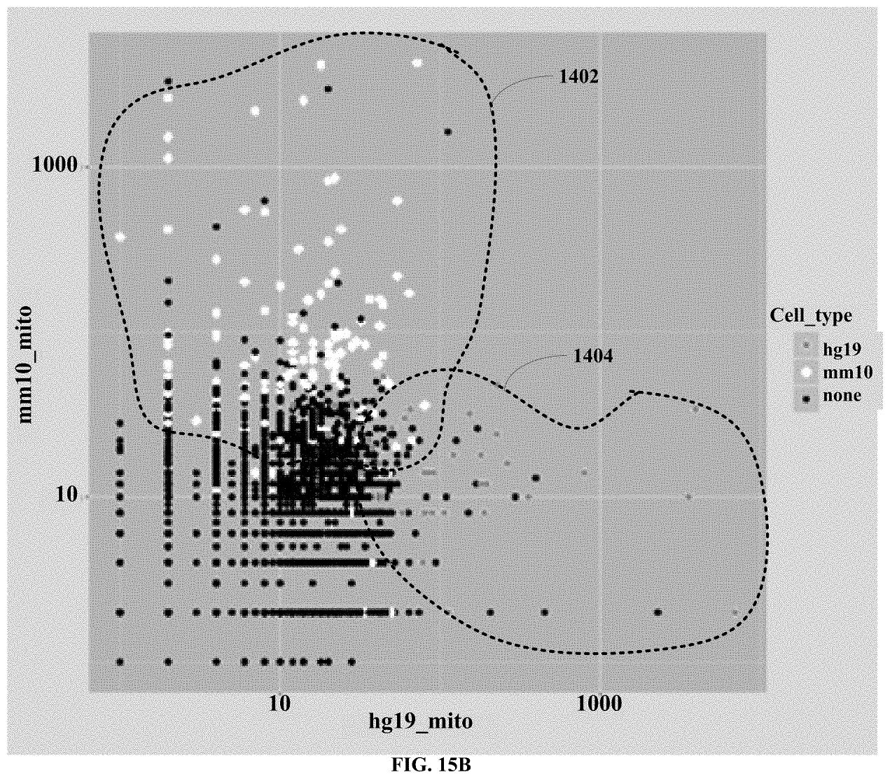

In some cases, sequences from mitochondrial nucleic acid may be obtained from the ATAC-seq methods disclosed herein. For example, mitochondrial DNA from a cell may associate with a nuclear membrane, such that nuclei enclosed in a partition may be attached to mitochondrial DNA from that same cell. In this case, sequences from mitochondrial DNA may be obtained and analyzed. Nucleic acid variants may be identified from mitochondrial DNA. Obtaining and analyzing mitochondrial nucleic acid sequences from a single cell may be useful in, for example, linear tracing, studying heteroplasmy, etc. To this end, FIGS. 14 and 15 demonstrate that the ATAC-seq data that is acquired using the disclosed methods from mitochondrial DNA exhibits single cell behavior, meaning that the barcoded nucleic acid fragments from such mitochondrial DNA in fact represent the exact cells form which such nucleic acid fragments originate. In other words, like the case where the nucleic acid fragments originate from genomic DNA within a nucleus, the barcodes for barcoded nucleic acid fragments that originate from mitochondrial DNA uniquely represent the cells from which they originate. FIGS. 14A and 14B show analysis of single cell sequencing data from a barnyard mixture of human GM12878 and mouse EL4 cells where about 40% of the barcoded nucleic acid fragments originate from mitochondria while the rest of the barcoded nucleic acid fragments originate from genomic nucleic acids. That is, for FIG. 14, the biological sample that is analyzed in accordance with the disclosed methods is a mixture of human GM12878 cells and mouse EL4 cells in which 40% of the barcoded nucleic acid fragments originate from human or mouse mitochondria. FIG. 14A shows analysis of all of the barcoded nucleic acid fragments. That is, all the measured barcoded nucleic acid fragments that have the same barcode are aligned to a human reference sequence that is combination human reference genome/human mitochondrial genome to receive a human alignment score (Y-axis) and are further aligned to a mouse reference sequence that is a combination mouse reference genome/mouse mitochondrial genome to receive a mouse alignment score (X-axis). In this way, for each barcode (which is supposed to represent a single cell), a pair of alignment scores is obtained, one against the human reference sequence and the other against the mouse reference sequence. Each pair of alignment scores is plotted as (X, Y coordinates) in FIG. 14. Finally, respective plotted points in FIG. 14 are shaded based on whether the measured barcoded nucleic acid fragments having the barcode represented by the respective point contain only human specific SNPs (light grey), only mouse specific SNPs (white grey), or did not exhibit mouse or human specific SNPs (black). If each point in FIG. 14 in fact exhibits single cell behavior, it is expected that the measured barcoded nucleic acid fragments containing only human specific SNPs will form one cluster and that the measured barcoded nucleic acid fragments containing only mouse specific SNPs will form another cluster. This would mean that the measured barcoded nucleic acid fragments having the same barcode in fact derive from a single species, and hence, in a barnyard sample, from a single cell. Such clustering is clearly demonstrated in FIG. 14A when all measured barcoded nucleic acid fragments are used to contribute to the coordinate calculations used to form the points in the graph. That is, it is seen that all barcodes that contain measured barcoded nucleic acid fragments that only have mouse SNPs tend to cluster into cluster 1402. Likewise, it is seen that all barcodes that contain measured barcoded nucleic acid fragments that only have human SNPs tend to cluster into cluster 1404. FIG. 14B shows analysis of the mitochondria barcoded nucleic acid fragments only. Because only mitochondria barcoded nucleic acid fragments are used, only the 12 SNPs detected in human mitochondria, and the 6 SNPs detected in mouse mitochondria from this analysis can be used to shade the points in the graph. Moreover, the alignments made for the respective points in FIG. 14B are made only against the human and mouse reference mitochondrial genomes, not the full genomes of human and mouse. Thus, only the barcoded nucleic acid fragments that are mitochondrial in origin are used to determine whether the points are capable of clustering based on the human and mouse SNPs in FIG. 14B. Despite the limitation that only barcoded nucleic acid fragments that are mitochondrial in origin are used in FIG. 14B, it is still seen that it is seen that all barcodes that contain measured barcoded nucleic acid fragments that only have mouse SNPs tend to cluster into cluster 1402 in FIG. 14B. Likewise, it is seen that all barcodes that contain measured barcoded nucleic acid fragments that only have human SNPs tend to cluster into cluster 1404 in FIG. 14B. FIGS. 15A and 15B show the same analysis as corresponding FIGS. 14A and 14B, except that now, only 10% of the measured barcoded nucleic acid fragments are mitochondrial in origin while the remaining measured barcoded nucleic acid fragments are human in origin. In this regard, FIG. 15A, like FIG. 14A, shows analysis of all measured barcoded nucleic acid fragments. FIG. 15B, like FIG. 15B, shows analysis of measured barcoded nucleic acid fragments of mitochondrial origin only.

Germline Variants Affect Chromatin Accessibility in scATAC-Seq.

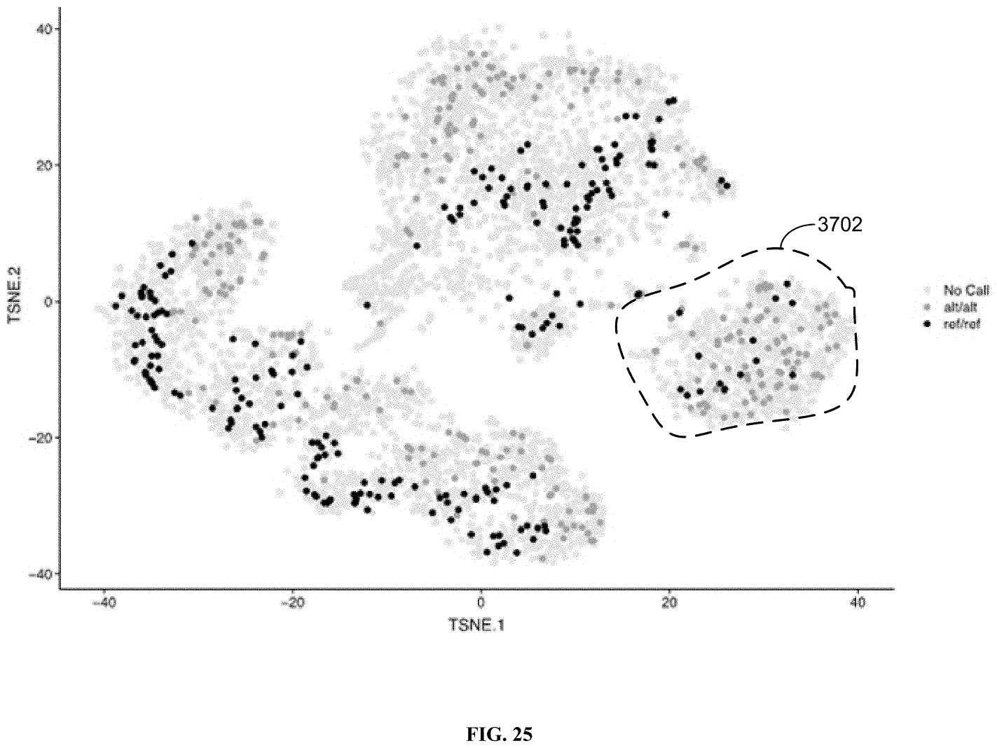

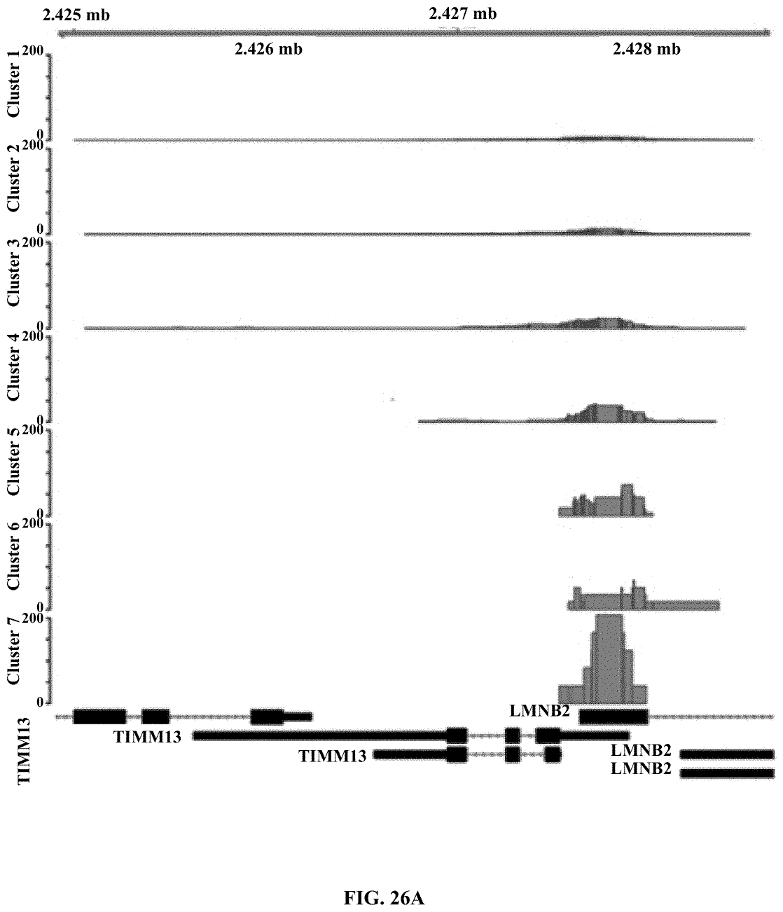

The disclosed systems and methods advantageously allow for the identification of germ line variants that affect chromatin accessibility, including regions that encompass transcription factor binding sites. FIG. 25 shows the results from a single cell sequencing experiment, where sequences from the peripheral blood mononuclear cells (PBMCs) cells of two subjects (Donor 1 and Donor 2) were mixed and analyzed for SNVs, such that SNVs specific to each individual were identified. FIG. 25 grey scale codes for a particular allelic position that is the site of a SNV in the vicinity of the human B type nuclear lamin (LMB2) gene in Donor 1. To produce the data shown in FIG. 25, a first pool of barcoded nucleic acid fragments was obtained from Donor 1 by a first procedure that comprises (i) generating, in each respective cell of a first plurality of cells obtained from Donor 1, a corresponding plurality of template nucleic acid fragments using a transposase-nucleic acid complex comprising a transposase molecule and a transposon end nucleic acid molecule in the respective cell. Further, a first plurality of partitions was created, where each respective partition in the first plurality of partitions comprises: (a) a respective single biological particle in the first plurality of cells, (b) the corresponding plurality of template nucleic acid fragments and (c) a corresponding plurality of nucleic acid barcode molecules comprising a corresponding common barcode sequence that is unique to the respective single cell. Further, a corresponding plurality of barcoded nucleic acid fragments was created, in each respective partition in the first plurality of partitions, using the corresponding plurality of nucleic acid barcode molecules and the corresponding plurality of template nucleic acid fragments within the respective partition, where the plurality of barcoded nucleic acid fragments in each respective partition in the first plurality of partitions collectively form the first pool of barcoded nucleic acid fragments in electronic form. The same procedure was performed using the plurality of cells obtained from Donor 2, thereby forming a second pool of barcoded nucleic acid fragments in electronic form derived from Donor 2.

Further, the first pool of barcoded nucleic acid fragments from Donor 1 was used to identify a first plurality of loci using a variant calling procedure (e.g, GATK4 or Mutect), and for each respective locus in the first plurality of loci, a corresponding set of alleles for the respective locus (e.g., using GATK4 or Mutect). This first plurality of loci thus represent the loci that have a SNP within Donor 1 cells.

Further, the second pool of barcoded nucleic acid fragments, from Donor 2, was used to identify a second plurality of loci, and for each respective locus in the second plurality of loci, a corresponding set of alleles for the respective locus (e.g., using GATK4 or Mutect). This second plurality of loci thus represent the loci that have a SNP within Donor 2 cells. The second plurality of loci are thus different than the first plurality of loci.

For each respective locus in both the first and second plurality of loci, a second procedure was performed that comprised i) identifying a corresponding subset of the first pool of barcoded nucleic acid fragments that map to the respective locus, ii) using an alignment of each respective barcoded nucleic acid fragment in the corresponding subset of the first pool of barcoded nucleic acid fragments to determine an allelic identity of each respective barcoded nucleic acid fragment from among the corresponding set of alleles for the respective locus, and iii) categorizing each respective barcoded nucleic acid fragment in the corresponding subset of the pool of barcoded nucleic acid fragments by the allelic identity of the respective barcoded nucleic acid fragment. Referring to FIG. 16, a corresponding genotypic data structure 1633 was constructed for each cell (biological particle) 1634 in the first plurality of cells (from Donor 1) using the barcode identities of the first pool of barcoded nucleic acid fragments and the identified alleles for each such barcoded nucleic acid fragment (from among the first and second plurality of loci) thereby constructing a first plurality of genotypic data structures, one for each cell in the first plurality of cells.

For each respective locus in both the first and second plurality of loci, a third procedure was performed that comprised i) identifying a corresponding subset of the second pool of barcoded nucleic acid fragments that map to the respective locus, ii) using an alignment of each respective barcoded nucleic acid fragment in the corresponding subset of the second pool of barcoded nucleic acid fragments to determine an allelic identity of each respective barcoded nucleic acid fragment from among the corresponding set of alleles for the respective locus, and iii) categorizing each respective barcoded nucleic acid fragment in the corresponding subset of the pool of barcoded nucleic acid fragments by the allelic identity of the respective barcoded nucleic acid fragment (from among the first and second plurality of loci). Referring to FIG. 16, a corresponding genotypic data structure 1633 was constructed for each cell 1634 in the second plurality of cells (from Donor 2) using the barcode identities of the second pool of barcoded nucleic acid fragments and the identified alleles for each such barcoded nucleic acid fragment thereby constructing a second plurality of genotypic data structures, one for each cell in the second plurality of cells.

The corresponding genotypic data structure for each cell in the first and second plurality of cells was used to segregate the cells to determine a property of each cell. Referring to FIG. 25, for each respective cell, a corresponding vector was generated whose elements comprised the allelic identity of the respective cell for each locus in the first and second plurality of loci. Referring to FIG. 16, one manner in which this is done is to call, for each locus 1630, the corresponding allele 1638 that was observed most often in the barcoded nucleic acid fragments attributed, through barcoding, to the respective cell. These vectors therefore represent the alleles of each respective cell in the first and second plurality of cells for each locus in the first and second plurality of loci in multidimensional space. These vectors were compressed to two dimensional space using t-SNE. As such, t-SNE used the corresponding genotypic data structure for each cell in the first and second plurality of cells to segregate the plurality of cells into two clusters, one of which represents Donor 1, and the other of which represents Donor 2.

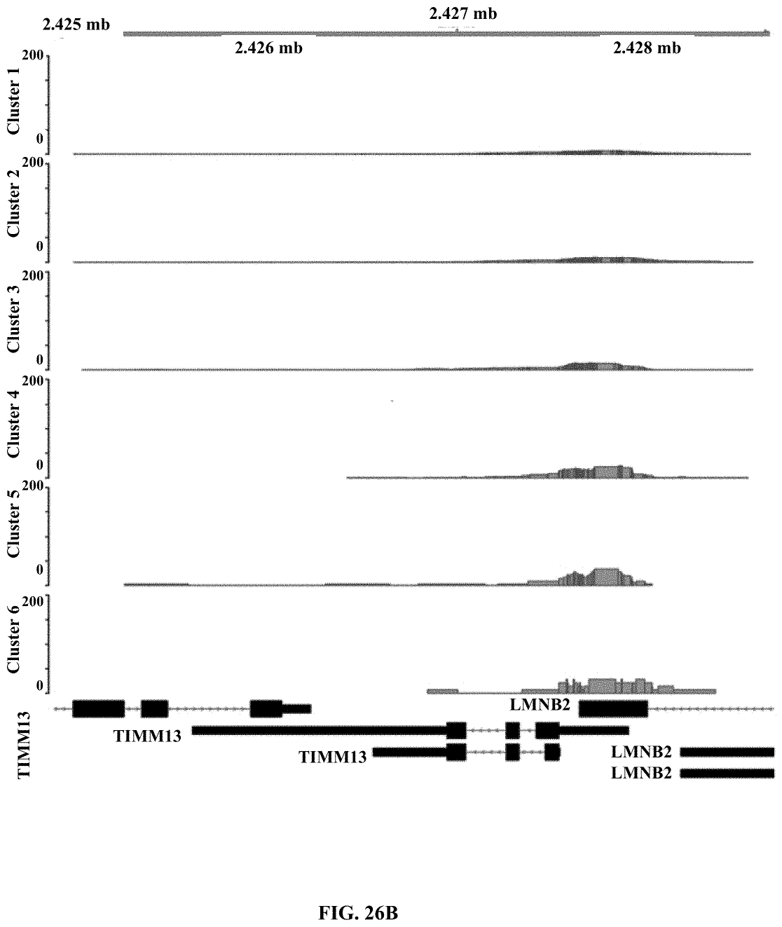

Turning to FIG. 25, the above described t-SNE process provides a “dot” for each respective vector. Each respective vector corresponds to the allelic calls across the first and second plurality of alleles for a corresponding cell. Thus, each “dot” in FIG. 25 represents a cell in the first (Donor 1) or second (Donor 2) plurality of cells. Next, each “dot” in FIG. 25 is grey-scale coded by the allelic call made for the corresponding cell at the genomic position for the LMB2 gene. Specifically, those cells that have the alternative allele for the LMB2 gene are shaded in dark grey, and originate from Donor 1. Those cells that have the wildtype (reference) allele for the LMB2 gene are shaded black, and originate from Donor 2. Those cells that do not make an allelic call for the LMB2 gene are shaded very light grey. FIG. 26A provides the allelic distribution for the locus of the LMB2 gene across the cells from Donor 2 (reference allele) that clustered into t-SNE cluster 3702 of FIG. 25. FIG. 26B provides the allelic distribution for the locus of the LMB2 gene across the cells from Donor 1 (alternate allele) that clustered into t-SNE cluster 3702 of FIG. 25. It is seen from a comparison of FIGS. 26A and 26B that the cells from Donor 2 have more barcoded nucleic acid fragments at this allele the cells from Donor 1, indicating that the chromatin accessibility for the LMB2 gene in the wildtype Donor 2 cells is more open than in Donor 1.

Kits.

Also provided herein are kits for analyzing the accessible chromatin (e.g., for ATAC-seq) and/or RNA transcripts of individual cells or small populations of cells. The kits may include one or more of the following: one, two, three, four, five or more, up to all of partitioning fluids, including both aqueous buffers and non-aqueous partitioning fluids or oils, nucleic acid barcode libraries that are releasably associated with beads, as described herein, microfluidic devices, reagents for disrupting cells amplifying nucleic acids, and providing additional functional sequences on fragments of cellular nucleic acids or replicates thereof, as well as instructions for using any of the foregoing in the methods described herein.

Computer Systems.

FIG. 16 is a block diagram illustrating an exemplary, non-limiting system for identifying a structural variation or a copy number variation in a biological sample of a subject in accordance with some implementations. The system 1600 in some implementations includes one or more processing units CPU(s) 1602 (also referred to as processors), one or more network interfaces 1604, a user interface 1606, a memory 1612, and one or more communication buses 1614 for interconnecting these components. One example of user interface 1606 is depicted in FIG. 9. The communication buses 1614 optionally include circuitry (sometimes called a chipset) that interconnects and controls communications between system components. The memory 1612 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, other random access solid state memory devices, or any other medium which can be used to store desired information; and optionally includes non-volatile memory, such as one or more magnetic disk storage devices, optical disk storage devices, flash memory devices, or other non-volatile solid state storage devices.

The memory 1612 optionally includes one or more storage devices remotely located from the CPU(s) 1602. The memory 1612, or alternatively the non-volatile memory device(s) within the memory 1612, comprises a non-transitory computer readable storage medium. In some implementations, the memory 1612 or alternatively the non-transitory computer readable storage medium stores the following programs, modules and data structures, or a subset thereof:
an optional operating system 1616, which includes procedures for handling various basic system services and for performing hardware dependent tasks;
an optional network communication module (or instructions) 1618 for connecting the device 1600 with other devices, or a communication network;
an optional characterization module 1620 for identifying a structural variation or a copy number variation in a subject;
a plurality of barcoded nucleic acid fragments 904-1, 904-2, and 904-M, inclusive, each respective barcoded nucleic acid fragment in the plurality of barcoded nucleic acid fragment derived using a biological sample and comprising at least a barcode 1624-1 and sequence encoding portion 1626-1, both of which are described in detail above;
a loci data structure 1628 comprising a plurality of loci 1630-1, 1630-2, and 1630-X, inclusive, each loci in the plurality of loci comprising a plurality of alleles 1632-1-1 and 1632-1-N, inclusive; and
a plurality of genotype data structures 1633, each respective genotypic data structure in the plurality of genotype data structures including, for a corresponding biological particle 1634 in the plurality of biological particles, for each respective locus 1630-1, 1630-2, and 1630-X inclusive, in a plurality of loci: (i) a coverage of the respective locus 1630 for the respective biological particle 1634 and (ii) a proportion 1638-1-1, . . . , 1638-1-N inclusive of each allele 1632 in the corresponding set of alleles for the respective locus 1630 observed for the biological particle 1634.

In some implementations, the user interface 1606 includes an input device (e.g., a keyboard, a mouse, a touchpad, a track pad, and/or a touch screen) 1610 for a user to interact with the system 1600 and a display 1608.

In some implementations, one or more of the above identified elements are stored in one or more of the previously mentioned memory devices, and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 1612 optionally stores a subset of the modules and data structures identified above. Furthermore, in some embodiments, the memory stores additional modules and data structures not described above. In some embodiments, one or more of the above identified elements is stored in a computer system, other than that of system 1600, that is addressable by system 1600 so that system 1600 may retrieve all or a portion of such data when needed.

Although FIG. 16 shows an exemplary system 1600, the figure is intended more as functional description of the various features that may be present in computer systems than as a structural schematic of the implementations described herein. In practice, and as recognized by those of ordinary skill in the art, items shown separately could be combined and some items could be separated.

### EXAMPLES

Although one or more of the Examples herein make use of partitions that comprise droplets in an emulsion (e.g., droplet emulsion partitions), any of the above-described partitions (such as wells) can be utilized in the methods, systems, and compositions described below. For a description of exemplary ATAC-seq methodologies, compositions, and systems, including single cell analyses, see, e.g., U.S. Pat. No. 10,059,989 and U.S. Pat. Pub. 20180340171, which are both hereby incorporated by reference in their entireties.

### Example 1. Generation of Barcoded Nucleic Acid Fragments Using Bulk Tagmentation and Barcoding by Ligation in Partitions

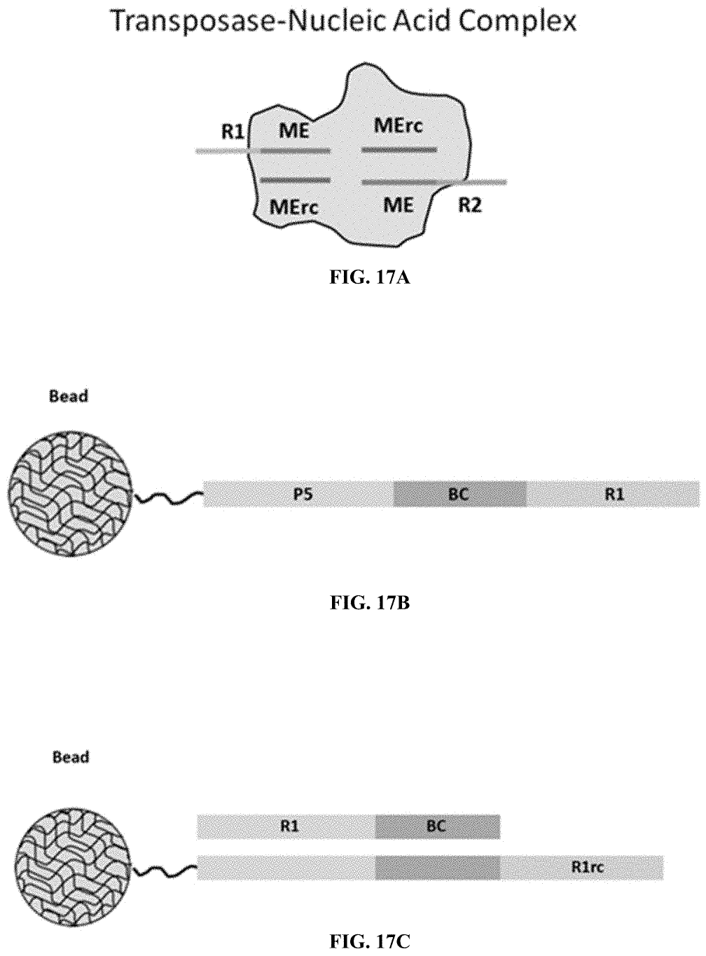

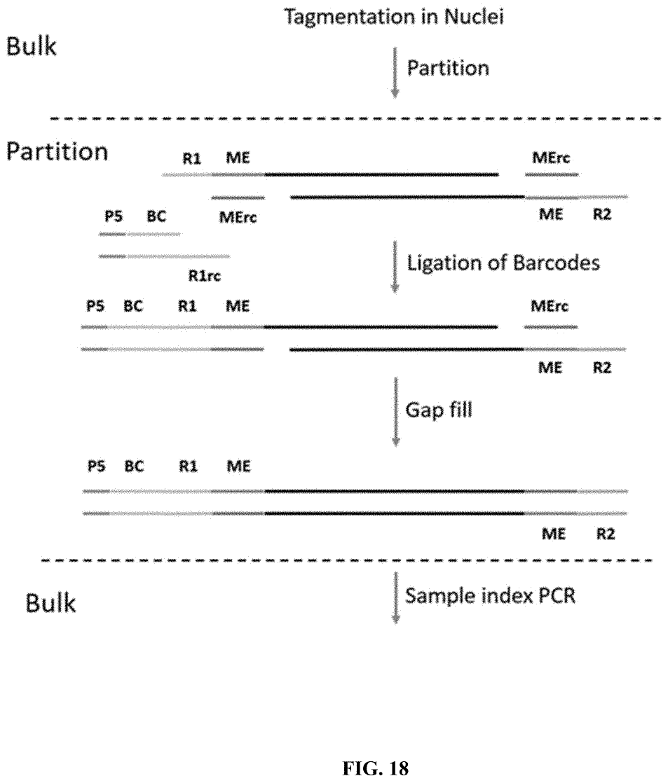

Intact nuclei are harvested in bulk from cells in a cell population of interest in a manner that substantially maintains native chromatin organization (e.g., using IGEPAL CA-630 mediated cell lysis). Nuclei are then incubated in the presence of a transposase-nucleic acid complex for adapter insertion. Alternatively, cells are permeable/permeabilized, allowing the transposase-nucleic acid complex to gain access to the nucleus. Although the transposase-nucleic acid complex can be prepared in a variety of different configurations, a transposase-nucleic acid complex comprising a transposase molecule (e.g., a transposase dimer) and two partially double-stranded adaptor oligonucleotides is illustrated in FIG. 17A. As shown in FIG. 17A, the transposase nucleic acid complex may comprise: (a) a first adapter oligonucleotide comprising a double stranded transposon end sequence (“ME”) and a single stranded Read1 sequencing primer sequence (“R1”); and (b) a second adapter oligonucleotide comprising a double stranded transposon end sequence (“ME”) and a single stranded Read2 sequencing primer sequence (“R2”). In some embodiments, the R1 and/or R2 sequencing primer in the first and/or second adapter oligonucleotide comprises a TruSeq R1 and/or R2 sequence, or a portion thereof. The transposase-nucleic acid complexes integrate the adaptors into the template nucleic acid and produce template nucleic acid fragments flanked by the partially double-stranded adaptors (“tagmentation”). See, e.g., FIG. 18. Because the transposase-nucleic acid complex preferably inserts on nucleosome-free regions of a template, the fragmented template nucleic acid fragments are representative of genome-wide areas of accessible chromatin. In some embodiments, the transposase molecules are inactivated prior to further processing steps.

Nuclei (or cells) comprising the adapter-flanked template nucleic acid fragments are then partitioned into a plurality of partitions (e.g., droplets or wells) such that at least some partitions comprise (1) a single nucleus (or cell) comprising the adapter-flanked template nucleic acid fragments; and (2) a plurality of partially double-stranded barcode oligonucleotide molecules comprising a doubled stranded barcode sequence (“BC”), a double-stranded P5 adapter sequence (“P5”), and a single stranded sequence complementary to the Read 1 sequence (“R1rc”). See, e.g., FIG. 18C. In some embodiments, the partially double-stranded barcode oligonucleotide molecules are attached to a solid or semi-solid particle (e.g., a bead, such as a gel bead) and partitioned such that at least some partitions (e.g., droplets or wells) comprise (1) a single nucleus (or cell) and (2) a single solid or semi-solid particle (e.g., bead, such as a gel bead). In addition to the aforementioned components, in some embodiments, the plurality of partitions (e.g., droplets or wells) further comprises reagents (e.g., enzymes and buffers) that facilitate the reactions described below.

Single cell- or nucleus-containing partitions (e.g., droplets or wells) are then subjected to conditions to release adapter-flanked template nucleic acid fragments from the nuclei (e.g., cell lysis). In certain embodiments, where barcode oligonucleotides (e.g., nucleic acid barcode molecules) are attached to a bead (e.g., gel bead), partitions (e.g., droplets or wells) are subjected to conditions to cause release of the barcode oligonucleotide molecules from the bead (e.g., gel bead) (e.g., depolymerization or degradation of the beads, for example, using a reducing agent such as DTT). After release from single nuclei, the adapter-flanked template nucleic acid fragments nay be subjected to conditions to phosphorylate the 5′ end of the Read1 sequence (e.g., using T4 polynucleotide kinase) for subsequent ligation steps. The barcode oligonucleotide molecules are then ligated onto the adapter-flanked template nucleic acid fragments using a suitable DNA ligase enzyme (e.g., T4 or E. coli DNA ligase) in a “sticky-end” ligation using the complementary Read1 sequences in the barcode oligonucleotides and the adapter-flanked template nucleic acid fragments. See FIG. 18.

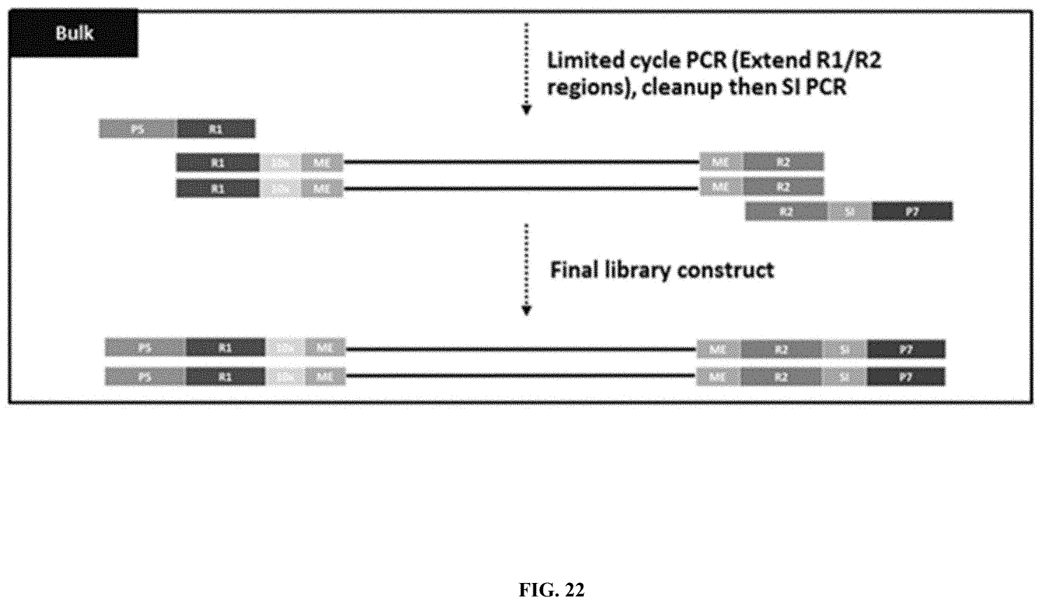

After barcode ligation, gaps remaining from the transposition reaction may be filled to produce barcoded, adapter-flanked template nucleic acid fragments. See FIG. 18. The barcoded, adapter-flanked template nucleic acid fragments are then released from the partitions (e.g., droplets or wells) and processed in bulk to complete library preparation for next generation high throughput sequencing (e.g., to add sample index (SI) sequences (e.g., “i7”) and/or further adapter sequences (e.g., “P7”)). See, e.g., FIG. 22. In alternative embodiments, the gap filling reaction is completed in bulk after barcoded, adapter-flanked template nucleic acid fragments have been released from the partitions (e.g., droplets or wells). The fully constructed library is then sequenced according to a suitable next-generation sequencing protocol (e.g., Illumina sequencing).

### Example 2. Generation of Barcoded Nucleic Acid Fragments Using Tagmentation and Barcoding by Ligation in Partitions

Cells from a cell population of interest (or nuclei from cells in a cell population of interest) are partitioned into a plurality of partitions (e.g., droplets or wells) such that at least some partitions comprise (1) a single cell (or a single nucleus) comprising a template nucleic acid; and (2) a plurality of partially double-stranded barcode oligonucleotide molecules (e.g., nucleic acid barcode molecules) comprising a doubled stranded barcode sequence (“BC”), a doubled stranded P5 adapter sequence (“P5”), and a single stranded sequence complementary to a Read 1 sequence (“R1rc”) (e.g., FIG. 17C). In some embodiments, the partially double-stranded barcode oligonucleotide molecules are attached to a solid or semi-solid particle (e.g., bead, such as a gel bead) and partitioned such that at least some partitions (e.g., droplets or wells) comprise (1) a single cell (or a single nucleus) and (2) a single solid or semi-solid particle (e.g., gel bead). In addition to the aforementioned components, in some embodiments, the plurality of partitions (e.g., droplets or wells) further comprises reagents (e.g., enzymes and buffers) that facilitate the reactions described below.

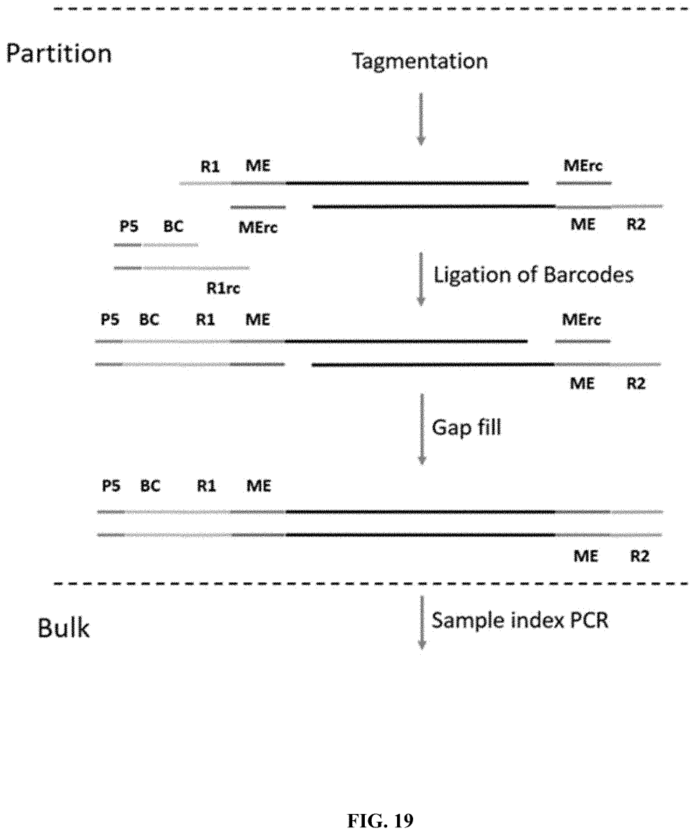

After partitioning into partitions (e.g., droplets or wells), the single cells (or nuclei) are lysed to release the template genomic DNA in a manner that substantially maintains native chromatin organization. Partitions (e.g., droplets or wells) are then subjected to conditions to generate a transposase-nucleic acid complex, e.g., as described in Example 1 and shown in FIG. 17A. Alternatively, in some embodiments, a plurality of pre-formed transposase-nucleic acid complexes as shown in, e.g., FIG. 17A are partitioned into the plurality of partitions (e.g., droplets or wells). Partitions (e.g., droplets or wells) are then subjected to conditions such that the transposase-nucleic acid complexes integrate the first and second adapter sequences into the template nucleic acid to generate double-stranded adapter-flanked template nucleic acid fragments. See FIG. 19. Because the transposase-nucleic acid complex preferably inserts on nucleosome-free regions of a template, the fragmented template nucleic acid fragments are representative of genome-wide areas of accessible chromatin. Alternatively, in some embodiments, the tagmentation reaction is performed in intact nuclei, and the nuclei are lysed after transposition to release the double-stranded adapter-flanked template nucleic acid fragments.

Samples are then processed generally as described in Example 1. In certain embodiments, where barcode oligonucleotides (e.g., nucleic acid barcode molecules) are attached to a solid or semi-solid particle (e.g., bead, such as a gel bead), partitions are subjected to conditions to cause release of the barcode oligonucleotide molecules from the solid or semi-solid particle (e.g., gel bead) (e.g., depolymerization or degradation of beads, for example, using a reducing agent such as DTT). In some embodiments, the transposase molecules are inactivated (e.g., by heat inactivation) prior to further processing steps. The adapter-flanked template nucleic acid fragments are subjected to conditions to phosphorylate the 5′ end of the Read1 sequence (e.g., using T4 polynucleotide kinase) of the adapter-flanked template nucleic acid fragments. After phosphorylation, the barcode oligonucleotide molecules are then ligated onto the adapter-flanked template nucleic acid fragments using a suitable DNA ligase enzyme (e.g., T4 or E. coli DNA ligase) in a “sticky-end” ligation using the complementary Read1 sequences in the barcode oligonucleotides and the adapter-flanked template nucleic acid fragments. See FIG. 18.

After barcode ligation, gaps remaining from the transposition reaction are filled to produce barcoded, adapter-flanked template nucleic acid fragments. See FIG. 18. The barcoded, adapter-flanked template nucleic acid fragments are then released from the partitions (e.g., droplets or wells) and processed in bulk to complete library preparation for next generation high throughput sequencing (e.g., to add sample index (SI) sequences (e.g., “i7”) and/or further adapter sequences (e.g., “P7”)). See, e.g., FIG. 22. In alternative embodiments, the gap filling reaction is completed in bulk after barcoded, adapter-flanked template nucleic acid fragments have been released from the droplets. The fully constructed library is then sequenced according to a suitable next-generation sequencing protocol (e.g., Illumina sequencing).

### Example 3. Generation of Barcoded Nucleic Acid Fragments Using Bulk Tagmentation and Barcoding by Linear Amplification in Partitions

Nuclei are harvested in bulk from cells in a cell population of interest in a manner that substantially maintains native chromatin organization. Alternatively, cells are permeabilized/permeable, allowing the transposase-nucleic acid complex to gain access to the nucleus. Nuclei (or permeabilized cells) are then incubated in the presence of a transposase-nucleic acid complex as described in Example 1. See FIG. 17A.

Nuclei (or cells) comprising the adapter-flanked template nucleic acid fragments are then partitioned into a plurality of partitions (e.g., droplets or wells) such that at least some partitions comprise (1) a single nucleus (or cell) comprising the adapter-flanked template nucleic acid fragments; and (2) a plurality of single-stranded barcode oligonucleotide molecules (e.g., nucleic acid barcode molecules) comprising a Read1 sequence (“R1”), or a portion thereof, a barcode sequence (“BC”), and a P5 adapter sequence (“P5”). See FIG. 17B. In some embodiments, the single-stranded barcode oligonucleotide molecules are attached to a solid or semi-solid particle (e.g., a bead, such as a gel bead) and partitioned such that at least some partitions (e.g., droplets or wells) comprise (1) a single nucleus (or cell) comprising the adapter-flanked template nucleic acid fragments and (2) a single solid or semi-solid particle (e.g., gel bead). In addition to the aforementioned components, in some embodiments, the plurality of partitions (e.g., droplets or wells) further comprises reagents (e.g., enzymes and buffers) that facilitate the reactions described below.

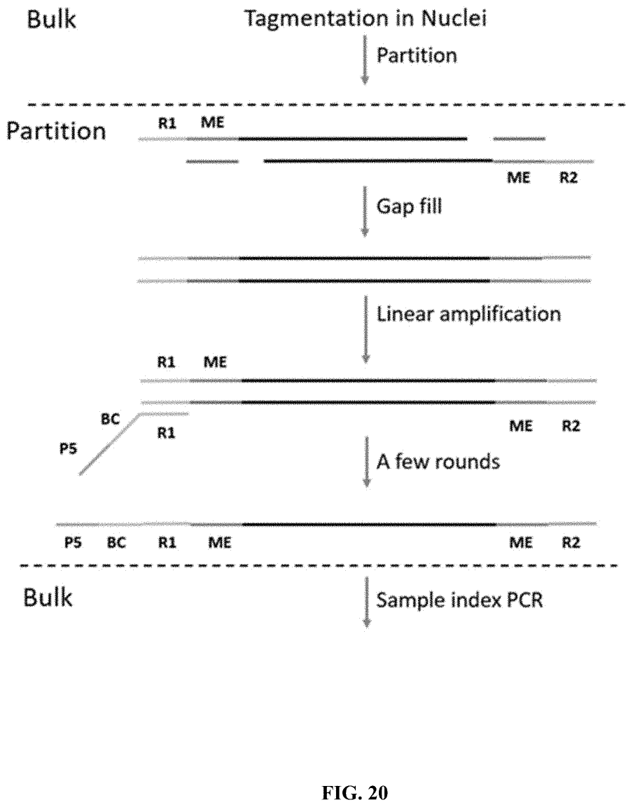

Single cell- or nucleus-containing partitions (e.g., droplets or wells) are then subjected to conditions to release the adapter-flanked template nucleic acid fragments from the nuclei. After the adapter-flanked template nucleic acid fragments are released, gaps from the transposition reaction are filled with a suitable enzyme. See FIG. 20. In certain embodiments, where barcode oligonucleotides (e.g., nucleic acid barcode molecules) are attached to a solid or semi-solid particle (e.g., bead, such as a gel bead), partitions (e.g., droplets or wells) are subjected to conditions to cause release of the barcode oligonucleotide molecules from the solid or semi-solid particle (e.g., bead, such as a gel bead) (e.g., depolymerization or degradation of beads, for example, using a reducing agent such as DTT). Gap-filled, adapter-flanked template nucleic acid fragments are then subjected to a linear amplification reaction using the single-stranded barcode oligonucleotide molecules as primers to produce barcoded, adapter-flanked template nucleic acid fragments. See FIG. 20.

The barcoded, adapter-flanked template nucleic acid fragments are then released from the partitions (e.g., droplets or wells) and processed in bulk to complete library preparation for next generation high throughput sequencing (e.g., to add sample index (SI) sequences (e.g., “i7”) and/or further adapter sequences (e.g., “P7”)). See, e.g., FIG. 22. The fully constructed library is then sequenced according to a suitable next-generation sequencing protocol (e.g., Illumina sequencing).

### Example 4. Generation of Barcoded Nucleic Acid Fragments Using Tagmentation and Barcoding by Linear Amplification in Partitions

Cells from a cell population of interest (or intact nuclei from cells in a cell population of interest) are partitioned into a plurality of partitions (e.g., droplets or wells) such that at least some partitions comprise (1) a single cell (or a single nucleus) comprising a template nucleic acid; and (2) a plurality of single-stranded barcode oligonucleotide molecules (e.g., nucleic acid barcode molecules) comprising a Read1 sequence (“R1”), a barcode sequence (“BC”), and a P5 adapter sequence (“P5”). See, e.g., FIG. 17B. In some embodiments, the single-stranded barcode oligonucleotide molecules are attached to a solid or semi-solid particle (e.g., bead, such as a gel bead) and partitioned such that at least some partitions (e.g., droplets or wells) comprise (1) a single cell (or a single nucleus) and (2) a single solid or semi-solid particle (e.g., bead, such as a gel bead). In addition to the aforementioned components, in some embodiments, the plurality of partitions (e.g., droplets or wells) further comprises reagents (e.g., enzymes and buffers) that facilitate the reactions described below.

After partitioning into partitions (e.g., droplets or wells), the single cells (or nuclei) are lysed to release the template genomic DNA in a manner that substantially maintains native chromatin organization. In certain embodiments, where barcode oligonucleotides (e.g., nucleic acid barcode molecules) are attached to a solid or semi-solid particle (e.g., bead, such as a gel bead), partitions (e.g., droplets or wells) are subjected to conditions to cause release of the barcode oligonucleotide molecules from the solid or semi-solid particle (e.g., bead, such as a gel bead) (e.g., depolymerization or degradation of beads, for example, using a reducing agent such as DTT). Partitions (e.g., droplets or wells) are then subjected to conditions to generate a transposase-nucleic acid complex, e.g., as described in Example 1 and shown in FIG. 17A. Alternatively, in some embodiments, a plurality of pre-formed transposase-nucleic acid complexes as shown in, e.g., FIG. 17A are partitioned into the plurality of partitions (e.g., droplets or wells). Partitions (e.g., droplets or wells) are then subjected to conditions such that the transposase-nucleic acid complexes integrate the first and second adapter sequences into the template nucleic acid to generate double-stranded adapter-flanked template nucleic acid fragments. Because the transposase-nucleic acid complex preferably inserts on nucleosome-free regions of a template, the fragmented template nucleic acid fragments are representative of genome-wide areas of accessible chromatin. Alternatively, in some embodiments, the tagmentation reaction is performed in intact nuclei, and the nuclei are lysed after transposition to release the double-stranded adapter-flanked template nucleic acid fragments.

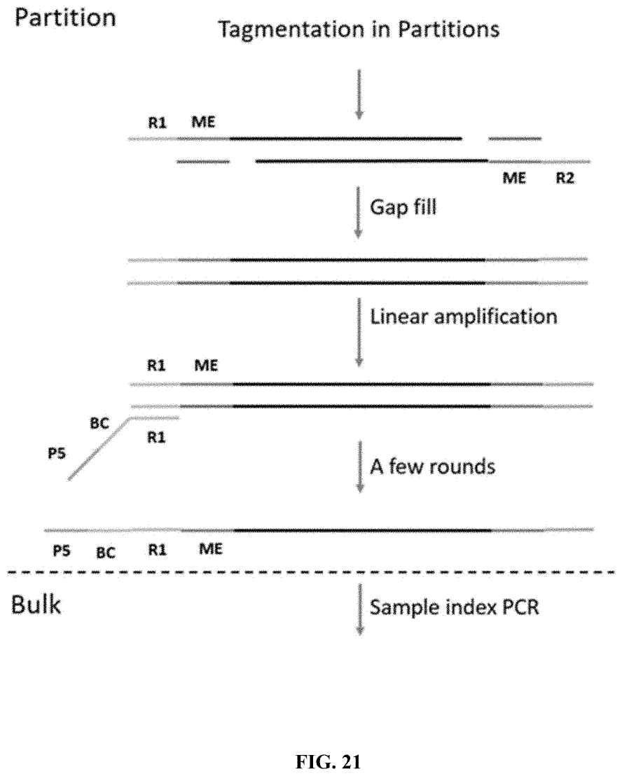

Samples are then processed generally as described in Example 11. After tagmentation, gaps from the transposition reaction are filled with a suitable gap-filling enzyme. See FIG. 21. Gap-filled adapter-flanked template nucleic acid fragments are then subjected to a linear amplification reaction using the single-stranded barcode oligonucleotide molecules as primers to produce barcoded, adapter-flanked template nucleic acid fragments. See FIG. 21.

The barcoded, adapter-flanked template nucleic acid fragments are then released from the partitions (e.g., droplets or wells) and processed in bulk to complete library preparation for next generation high throughput sequencing (e.g., to add sample index (SI) sequences (e.g., “i7”) and/or further adapter sequences (e.g., “P7”)). See, e.g., FIG. 22. The fully constructed library is then sequenced according to a suitable next-generation sequencing protocol (e.g., Illumina sequencing).

### Example 5. Use of a Classifier to Determine Physical State

Another aspect of the present disclosure is a physiological state determination method in which a pool of barcoded nucleic acid fragments is generated by a first procedure that comprises generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle. Further, a plurality of partitions is generated. Each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle. A corresponding plurality of barcoded nucleic acid fragments, in each respective partition in the plurality of partitions is generated, using the corresponding plurality of nucleic acid barcode molecules and the corresponding plurality of template nucleic acid fragments within the respective partition. The plurality of barcoded nucleic acid fragments in each respective partition in the plurality of partitions collectively form the pool of barcoded nucleic acid fragments in electronic form. A computer system comprising at least one processor and a memory storing at least one program for execution by the at least one processor, the at least one program comprising instructions for performing a second procedure is provided. In the second procedure, for each respective locus in a plurality of loci, a corresponding subset of the pool of barcoded nucleic acid fragments that map to the respective locus is identified. Further, an alignment of each respective barcoded nucleic acid fragment in the corresponding subset of the pool of barcoded nucleic acid fragments is done to determine an allelic identity of each respective barcoded nucleic acid fragment from among a corresponding set of alleles for the respective locus. Further still, each respective barcoded nucleic acid fragment in the corresponding subset of the pool of barcoded nucleic acid fragments is categorized by the allelic identity and barcode identity of the respective barcoded nucleic acid fragment, thereby determining a corresponding allelic distribution at each respective locus in the plurality of loci, for each biological particle in the plurality of biological particles. The corresponding allelic distribution includes an abundance of each allele in the corresponding set of alleles for the respective locus. The corresponding allelic distribution at each respective locus in the plurality of loci is used to determine the physiological state of the single test subject.

In some embodiments, a respective locus in the plurality of loci is biallelic and the corresponding set of alleles for the respective locus consists of a first allele and a second allele.

In some embodiments, the respective locus includes a heterozygous single nucleotide polymorphism (SNP), a heterozygous single nucleotide variant (SNV), a heterozygous insert, a heterozygous deletion, or a copy number variation.

In some embodiments, the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments.

In some embodiments, the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments.

In some embodiments, the plurality of loci comprises between two and 100 loci, more than 10 loci, more than 100 loci, or more than 500 loci.

In some embodiments, the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

In some embodiments, the corresponding common barcode sequence is localized to a contiguous set of oligonucleotides within the respective barcoded nucleic acid fragment.

In some embodiments, plurality of loci are identified by retrieving the plurality of loci and each corresponding set of alleles from a lookup table, file or data structure. In some alternative embodiments, the pool of barcoded nucleic acid fragments is used to identify the plurality of loci, and for each respective locus in the plurality of loci, the corresponding set of alleles for the respective locus.

In some embodiments, the alignment is a local alignment (e.g., a Smith-Waterman alignment) that aligns the respective barcoded nucleic acid fragment to a reference sequence using a scoring system that (i) penalizes a mismatch between a nucleotide in the respective barcoded nucleic acid fragment and a corresponding nucleotide in the reference sequence (e.g., all or portion of a reference genome) in accordance with a substitution matrix and (ii) penalizes a gap introduced into an alignment of the respective barcoded nucleic acid fragment and the reference sequence.

In some embodiments, the plurality of loci include one or more loci on a first chromosome and one or more loci on a second chromosome other than the first chromosome.

In some embodiments, each partition in the plurality of partitions is a droplet or a well.

In some embodiments, each biological particle in the plurality of biological particles is a single cell nuclei harvested from its cell. In some embodiments, each biological particle in the plurality of biological particles is a single cell.

In some embodiments, the transposase molecule is a native Tn5 transposase, a mutated hyperactive Tn5 transposase, or a Mu transposase. In some embodiments, the transposon end nucleic acid molecule is a Tn5 or modified Tn5 transposon end sequence.

In some embodiments, where the corresponding plurality of nucleic acid barcode molecules are attached to a solid or semi-solid particle (e.g., a gel bead).

In some embodiments, the physiological state is absence or presence of a disease. In some embodiments, the physiological state is a stage of a disease.

In some embodiments, the plurality of loci are in a reference genome (e.g., a human reference genome). In some embodiments, the reference genome is a mitochondrial genome.

In some embodiments, the corresponding allelic distribution at each respective locus in the plurality of loci is inputted into a classifier and the classifier, responsive to this inputting, provides the physiological state of the single test subject.

In some embodiments, the classifier is a multinomial classifier that provides a plurality of likelihoods, where each respective likelihood in the plurality of likelihoods is a likelihood that the single test subject has a corresponding physiological state in a plurality of physiological states. In some embodiments, each physiological state in the plurality of physiological states is a cancer class in a plurality of cancer classes.

In some embodiments, the classifier is a multivariate logistic regression algorithm. Logistic regression algorithms are disclosed in Agresti, An Introduction to Categorical Data Analysis, 1996, Chapter 5, pp. 103-144, John Wiley & Son, New York, which is hereby incorporated by reference.

In some embodiments, the classifier is a neural network algorithm or a convolutional neural network algorithm. Neural network algorithms, including convolutional neural network algorithms, are disclosed in See, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference.

In some embodiments, the classifier is a support vector machine (SVM) algorithm. SVM algorithms are described in Cristianini and Shawe-Taylor, 2000, “An Introduction to Support Vector Machines,” Cambridge University Press, Cambridge; Boser et al., 1992, “A training algorithm for optimal margin classifiers,” in Proceedings of the 5^{th} Annual ACM Workshop on Computational Learning Theory, ACM Press, Pittsburgh, Pa., pp. 142-152; Vapnik, 1998, Statistical Learning Theory, Wiley, New York; Mount, 2001, Bioinformatics: sequence and genome analysis, Cold Spring Harbor Laboratory Press, Cold Spring Harbor, N.Y.; Duda, Pattern Classification, Second Edition, 2001, John Wiley & Sons, Inc., pp. 259, 262-265; and Hastie, 2001, The Elements of Statistical Learning, Springer, New York; and Furey et al., 2000, Bioinformatics 16, 906-914, each of which is hereby incorporated by reference in its entirety. When used for classification, SVMs separate a given set of binary labeled data training set with a hyper-plane that is maximally distant from the labeled data. For cases in which no linear separation is possible, SVMs can work in combination with the technique of “kernels,” which automatically realizes a non-linear mapping to a feature space. The hyper-plane found by the SVM in feature space corresponds to a non-linear decision boundary in the input space.

In some embodiments, the classifier is a decision tree algorithm. Decision trees are described generally by Duda, 2001, Pattern Classification, John Wiley & Sons, Inc., New York, pp. 395-396, which is hereby incorporated by reference. Tree-based methods partition the feature space into a set of rectangles, and then fit a model (like a constant) in each one. In some embodiments, the decision tree is random forest regression. One specific algorithm that can be used is a classification and regression tree (CART). Other specific decision tree algorithms include, but are not limited to, ID3, C4.5, MART, and Random Forests. CART, ID3, and C4.5 are described in Duda, 2001, Pattern Classification, John Wiley & Sons, Inc., New York, pp. 396-408 and pp. 411-412, which is hereby incorporated by reference. CART, MART, and C4.5 are described in Hastie et al., 2001, The Elements of Statistical Learning, Springer-Verlag, New York, Chapter 9, which is hereby incorporated by reference in its entirety. Random Forests are described in Breiman, 1999, “Random Forests—Random Features,” Technical Report 567, Statistics Department, U.C. Berkeley, September 1999, which is hereby incorporated by reference in its entirety.

In some embodiments, the classifier is a clustering algorithm. Clustering is described at pages 211-256 of Duda and Hart, Pattern Classification and Scene Analysis, 1973, John Wiley & Sons, Inc., New York, (hereinafter “Duda 1973”) which is hereby incorporated by reference in its entirety. As described in Section 6.7 of Duda 1973, the clustering problem is described as one of finding natural groupings in a dataset. To identify natural groupings, two issues are addressed. First, a way to measure similarity (or dissimilarity) between two samples is determined. This metric (similarity measure) is used to ensure that the samples in one cluster are more like one another than they are to samples in other clusters. Second, a mechanism for partitioning the data into clusters using the similarity measure is determined.

Similarity measures are discussed in Section 6.7 of Duda 1973, where it is stated that one way to begin a clustering investigation is to define a distance function and to compute the matrix of distances between all pairs of samples in the training set. If distance is a good measure of similarity, then the distance between reference entities in the same cluster will be significantly less than the distance between the reference entities in different clusters. However, as stated on page 215 of Duda 1973, clustering does not require the use of a distance metric. For example, a nonmetric similarity function s(x, x′) can be used to compare two vectors x and x′. Conventionally, s(x, x′) is a symmetric function whose value is large when x and x′ are somehow “similar.” An example of a nonmetric similarity function s(x, x′) is provided on page 218 of Duda 1973.

In some embodiments, the classifier is a Naive Bayes algorithm. See, for example, Caruana and Niculescu-Mizil, 2006, “An empirical comparison of supervised learning algorithms,” Proc. 23rd International Conference on Machine Learning, which is hereby incorporated by reference.

In some embodiments, the classifier is a nearest neighbor algorithm. See, for example, Gutin et al., 2002, “Traveling salesman should not be greedy: domination analysis of greedy-type heuristics for the TSP,” Discrete Applied Mathematics 117, pp. 81-86, which is hereby incorporated by reference.

In some embodiments, the classifier is a random forest algorithm. Random Forests are described in Breiman, 1999, “Random Forests—Random Features,” Technical Report 567, Statistics Department, U.C. Berkeley, September 1999, which is hereby incorporated by reference in its entirety.

In some embodiments, the classifier is a boosted trees algorithm. See, for example, Kégl and Balázs, 2013, “The return of AdaBoost.MH: multi-class Hamming trees,” arXiv:1312.6086 and S̆ochman et al., 2004, “Adaboost with Totally Corrective Updates for Fast Face Detection,” ISBN 978-0-7695-2122-0, each of which is hereby incorporated by reference.

### REFERENCES CITED AND ALTERNATIVE EMBODIMENTS

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The present invention can be implemented as a computer program product that comprises a computer program mechanism embedded in a nontransitory computer readable storage medium. For instance, the computer program product could contain the program modules shown in FIG. 16, and/or described in FIG. 16 or 24. These program modules can be stored on a CD-ROM, DVD, magnetic disk storage product, USB key, or any other non-transitory computer readable data or program storage product.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed:

1. A structural variation identification method comprising:
A) generating a pool of barcoded nucleic acid fragments by a first procedure that comprises:
(i) generating, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
at a computer system comprising at least one processor and a memory storing at least one program for execution by the at least one processor, the at least one program comprising instructions for:
B) identifying a plurality of loci, and for each respective locus in the plurality of loci, a corresponding set of alleles for the respective locus;
C) for each respective locus in the plurality of loci, performing a second procedure that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
D) using the corresponding allelic distribution at each respective locus in the plurality of loci to identify a structural variation within a biological particle in the plurality of biological particles.

2. The method of claim 1, wherein a respective locus in the plurality of loci is biallelic and the corresponding set of alleles for the respective locus consists of a first allele and a second allele.

3. The method of claim 1, wherein the structural variation is a heterozygous single nucleotide polymorphism (SNP), a heterozygous single nucleotide variant (SNV), a heterozygous insert, a heterozygous deletion, or a copy number variation at a locus in the plurality of loci.

4. The method of claim 1, wherein the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments.

5. The method of claim 1, wherein the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments.

6. The method of claim 1, wherein the plurality of loci comprises between two and 100 loci, more than 10 loci, more than 100 loci, or more than 500 loci.

7. The method of claim 1, wherein the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

8. The method of claim 1, wherein the corresponding common barcode sequence is localized to a contiguous set of oligonucleotides within the respective barcoded nucleic acid fragment.

9. The method of claim 8, wherein the contiguous set of oligonucleotides is an N-mer, wherein N is an integer selected from the set {4, . . . , 20}.

10. The method of claim 1, wherein the B) identifying the plurality of loci comprises retrieving the plurality of loci and each corresponding set of alleles from a lookup table, file or data structure.

11. The method of claim 1, wherein the pool of barcoded nucleic acid fragments is used to identify the plurality of loci, and for each respective locus in the plurality of loci, the corresponding set of alleles for the respective locus.

12. The method of claim 1, wherein the alignment is a local alignment that aligns the respective barcoded nucleic acid fragment to a reference sequence using a scoring system that (i) penalizes a mismatch between a nucleotide in the respective barcoded nucleic acid fragment and a corresponding nucleotide in the reference sequence in accordance with a substitution matrix and (ii) penalizes a gap introduced into an alignment of the respective barcoded nucleic acid fragment and the reference sequence.

13. The method of claim 12, wherein the local alignment is a Smith-Waterman alignment.

14. The method of claim 12, wherein the reference sequence is all or portion of a reference genome.

15. The method of claim 1, the method further comprising removing from the pool of barcoded nucleic acid fragments one or more barcoded nucleic acid fragments that do not overlay any loci in the plurality of loci.

16. The method of claim 1, wherein the plurality of loci include one or more loci on a first chromosome and one or more loci on a second chromosome other than the first chromosome.

17. The method of claim 1, wherein each partition in the plurality of partitions is a droplet or a well.

18. The method of claim 1, wherein each biological particle in the plurality of biological particles is a single cell nuclei harvested from its cell.

19. The method of claim 1, wherein each biological particle in the plurality of biological particles is a single cell.

20. The method of claim 1, wherein the transposase molecule is a native Tn5 transposase, a mutated hyperactive Tn5 transposase, or a Mu transposase.

21. The method of claim 1, wherein the transposon end nucleic acid molecule is a Tn5 or modified Tn5 transposon end sequence.

22. The method of claim 1, wherein the corresponding plurality of nucleic acid barcode molecules are attached to a solid or semi-solid particle.

23. The method of claim 22, wherein the solid or semi-solid particle is a gel bead.

24. The method of claim 1, wherein the biological sample is from a single subject.

25. The method of claim 1, wherein the biological sample is from a plurality of subjects.

26. The method of claim 1, wherein the using D) determines a corresponding genotypic data structure for each biological particle in the plurality of biological particles, thereby constructing a plurality of genotypic data structures and wherein the at least one program further comprises using the corresponding genotypic data structure for each biological particle in the plurality of particles to segregate the plurality of biological particles to determine a property of each biological particle in the plurality of biological particles.

27. The method of claim 26, wherein the property is absence or presence of a disease.

28. The method of claim 26, wherein the property is a stage of a disease.

29. The method of claim 26, wherein the property is a cell type.

30. The method of claim 26, wherein the property is an identification of a species.

31. The method of claim 1, wherein the plurality of loci are in a reference genome.

32. The method of claim 31, wherein the reference genome is a human reference genome.

33. The method of claim 31, wherein the reference genome is a mitochondrial genome.

34. The method of claim 12, wherein the reference genome is a mitochondrial genome.

35. An electronic device, comprising:
one or more processors;
memory; and
one or more programs, wherein the one or more programs are stored in the memory and configured to be executed by the one or more processors, the one or more programs for identifying a structural variation, the one or more programs including instructions for:
A) obtaining, in electronic form, a pool of barcoded nucleic acid fragments by a first procedure that comprises:
(i) generating, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
B) identifying a plurality of loci, and for each respective locus in the plurality of loci, a corresponding set of alleles for the respective locus;
C) for each respective locus in the plurality of loci, performing a second procedure that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
D) using the corresponding allelic distribution at each respective locus in the plurality of loci to identify a structural variation within a biological particle in the plurality of biological particles.

36. A computer readable storage medium storing one or more programs, the one or more programs comprising instructions, which when executed by an electronic device with one or more processors and a memory cause the electronic device to identify a structural variation by a method comprising:
A) obtaining, in electronic form, a pool of barcoded nucleic acid fragments by a first procedure that comprises:
(i) generating, in each respective biological particle of a plurality of biological particles obtained from a biological sample, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
B) identifying a plurality of loci, and for each respective locus in the plurality of loci, a corresponding set of alleles for the respective locus;
C) for each respective locus in the plurality of loci, performing a second procedure that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among the corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
D) using the corresponding allelic distribution at each respective locus in the plurality of loci to identify a structural variation within a biological particle in the plurality of biological particles.

37. A physiological state determination method comprising:
A) obtaining a pool of barcoded nucleic acid fragments generated by a first procedure that comprises:
(i) generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
at a computer system comprising at least one processor and a memory storing at least one program for execution by the at least one processor, the at least one program comprising instructions for:
B) performing a second procedure, for each respective locus in a plurality of loci, that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among a corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
C) using the corresponding allelic distribution at each respective locus in the plurality of loci to determine the physiological state of the single test subject.

38. The method of claim 37, the method further comprising, prior the B) performing, identifying the plurality of loci, and for each respective locus in the plurality of loci, the corresponding set of alleles for the respective locus.

39. The method of claim 37, wherein a respective locus in the plurality of loci is biallelic and the corresponding set of alleles for the respective locus consists of a first allele and a second allele.

40. The method of claim 37, wherein the respective locus includes a heterozygous single nucleotide polymorphism (SNP), a heterozygous single nucleotide variant (SNV), a heterozygous insert, a heterozygous deletion, or a copy number variation.

41. The method of claim 37, wherein the corresponding plurality of barcoded nucleic acid fragments comprises 10,000 or more corresponding plurality of barcoded nucleic acid fragments, 50,000 or more corresponding plurality of barcoded nucleic acid fragments, 100,000 or more corresponding plurality of barcoded nucleic acid fragments, or 1×10⁶ or more corresponding plurality of barcoded nucleic acid fragments.

42. The method of claim 37, wherein the corresponding subset of the pool of barcoded nucleic acid fragments that map to the respective loci comprises 5 or more barcoded nucleic acid fragments, 100 or more barcoded nucleic acid fragments, or 1000 or more barcoded nucleic acid fragments.

43. The method of claim 37, wherein the plurality of loci comprises between two and 100 loci, more than 10 loci, more than 100 loci, or more than 500 loci.

44. The method of claim 37, wherein the corresponding common barcode sequence encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}.

45. The method of claim 37, wherein the corresponding common barcode sequence is localized to a contiguous set of oligonucleotides within the respective barcoded nucleic acid fragment.

46. The method of claim 37, wherein the identifying the plurality of loci comprises retrieving the plurality of loci and each corresponding set of alleles from a lookup table, file or data structure.

47. The method of claim 38, wherein the pool of barcoded nucleic acid fragments is used to identify the plurality of loci, and for each respective locus in the plurality of loci, the corresponding set of alleles for the respective locus.

48. The method of claim 37, wherein the alignment is a local alignment that aligns the respective barcoded nucleic acid fragment to a reference sequence using a scoring system that (i) penalizes a mismatch between a nucleotide in the respective barcoded nucleic acid fragment and a corresponding nucleotide in the reference sequence in accordance with a substitution matrix and (ii) penalizes a gap introduced into an alignment of the respective barcoded nucleic acid fragment and the reference sequence.

49. The method of claim 48, wherein the local alignment is a Smith-Waterman alignment.

50. The method of claim 48, wherein the reference sequence is all or portion of a reference genome.

51. The method of claim 37, wherein the plurality of loci include one or more loci on a first chromosome and one or more loci on a second chromosome other than the first chromosome.

52. The method of claim 37, wherein each partition in the plurality of partitions is a droplet or a well.

53. The method of claim 37, wherein each biological particle in the plurality of biological particles is a single cell nuclei harvested from its cell.

54. The method of claim 37, wherein each biological particle in the plurality of biological particles is a single cell.

55. The method of claim 37, wherein the transposase molecule is a native Tn5 transposase, a mutated hyperactive Tn5 transposase, or a Mu transposase.

56. The method of claim 37, wherein the transposon end nucleic acid molecule is a Tn5 or modified Tn5 transposon end sequence.

57. The method of claim 37, wherein the corresponding plurality of nucleic acid barcode molecules are attached to a solid or semi-solid particle.

58. The method of claim 57, wherein the solid or semi-solid particle is a gel bead.

59. The method of claim 37, wherein the physiological state is absence or presence of a disease.

60. The method of claim 37, wherein the physiological state is a stage of a disease.

61. The method of claim 37, wherein the plurality of loci are in a reference genome.

62. The method of claim 61, wherein the reference genome is a human reference genome.

63. The method of claim 61, wherein the reference genome is a mitochondrial genome.

64. The method of claim 37, wherein the using C) inputs the corresponding allelic distribution at each respective locus in the plurality of loci into a classifier, wherein the classifier responsive to this inputting provides the physiological state of the single test subject.

65. The method of claim 64, wherein the classifier is a multinomial classifier that provides a plurality of likelihoods, wherein each respective likelihood in the plurality of likelihoods is a likelihood that the single test subject has a corresponding physiological state in a plurality of physiological states.

66. The method of claim 65, wherein each physiological state in the plurality of physiological states is a cancer class in a plurality of cancer classes.

67. The method of claim 64, wherein the classifier is a multivariate logistic regression algorithm, a neural network algorithm, or a convolutional neural network algorithm.

68. The method of claim 64, wherein the classifier is a neural network algorithm, a support vector machine algorithm, a Naive Bayes algorithm, a nearest neighbor algorithm, a boosted trees algorithm, a random forest algorithm, a convolutional neural network algorithm, a decision tree algorithm, a regression algorithm, or a clustering algorithm.

69. An electronic device, comprising:
one or more processors;
memory; and
one or more programs, wherein the one or more programs are stored in the memory and configured to be executed by the one or more processors, the one or more programs for determining a physiological state, the one or more programs including instructions for:
A) obtaining, in electronic form, a pool of barcoded nucleic acid fragments generated by a first procedure that comprises:
(i) generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
B) performing a second procedure, for each respective locus in a plurality of loci, that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among a corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
C) using the corresponding allelic distribution at each respective locus in the plurality of loci to determine the physiological state of the single test subject.

70. A computer readable storage medium storing one or more programs, the one or more programs comprising instructions, which when executed by an electronic device with one or more processors and a memory cause the electronic device to determine a physiological by a method comprising:
A) obtaining, in electronic form, a pool of barcoded nucleic acid fragments generated by a first procedure that comprises:
(i) generating, in each respective biological particle of a plurality of biological particles obtained from a biological sample from a single test subject, a corresponding plurality of template nucleic acid fragments using a transposase-nucleic acid complex comprising a transposase molecule and a transposon end nucleic acid molecule in the respective biological particle,
(ii) generating a plurality of partitions, wherein each respective partition in the plurality of partitions comprises: (a) a respective single biological particle in the plurality of biological particles, (b) the corresponding plurality of template nucleic acid fragments and (c) a corresponding plurality of nucleic acid barcode molecules comprising a corresponding common barcode sequence that is unique to the respective single biological particle, and
(iii) generating a corresponding plurality of barcoded nucleic acid fragments, in each respective partition in the plurality of partitions, using the corresponding plurality of nucleic acid barcode molecules and the corresponding plurality of template nucleic acid fragments within the respective partition, wherein the plurality of barcoded nucleic acid fragments in each respective partition in the plurality of partitions collectively form the pool of barcoded nucleic acid fragments in electronic form;
B) performing a second procedure, for each respective locus in a plurality of loci, that comprises:
i) identifying a corresponding subset of the pool of barcoded nucleic acid fragments that map to the respective locus,
ii) using an alignment of each respective barcoded nucleic acid fragment in the corresponding subset of the pool of barcoded nucleic acid fragments to determine an allelic identity of each respective barcoded nucleic acid fragment from among a corresponding set of alleles for the respective locus, and
iii) categorizing each respective barcoded nucleic acid fragment in the corresponding subset of the pool of barcoded nucleic acid fragments by the allelic identity and barcode identity of the respective barcoded nucleic acid fragment, thereby determining a corresponding allelic distribution at each respective locus in the plurality of loci, for each biological particle in the plurality of biological particles, wherein the corresponding allelic distribution includes an abundance of each allele in the corresponding set of alleles for the respective locus; and
C) using the corresponding allelic distribution at each respective locus in the plurality of loci to determine the physiological state of the single test subject.
